SEQUENCE LISTING (98 sequences)

SEQ ID NO: 1 - DNA, 24 bp, Artificial Sequence
  Synthetic Polynucleotide
  misc_feature (1)..(24): Modified with a phosphorothioate bond
  misc_feature (24)..(24): Modified with /iSp18//iSp18//Chol/
tcgtcgttttgtcgttttgtcgtt

SEQ ID NO: 2 - RNA, 20 nt, Artificial Sequence
  Synthetic Polynucleotide
  misc_feature (1)..(1): Modified with /Chol//iSp18//iSp18/
  misc_feature (1)..(20): Modified with a phosphorothioate bond
uuguuguuguuguuguuguu

SEQ ID NO: 3 - RNA, 18 nt, Artificial Sequence
  Synthetic Polynucleotide
guccuucaaguccuucaa

SEQ ID NO: 4 - DNA, 25 bp, Artificial Sequence
  Synthetic Polynucleotide
tcgtcgtcgttcgaacgacgttgat

SEQ ID NO: 5 - DNA, 15 bp, Artificial Sequence
  Synthetic Polynucleotide
tcctggaggggttgt

SEQ ID NO: 6 - DNA, 18 bp, Artificial Sequence
  Synthetic Polynucleotide
tgctcctggaggggttgt

SEQ ID NO: 7 - DNA, 13 bp, Artificial Sequence
  Synthetic Polynucleotide
tgctggatgggaa

SEQ ID NO: 8 - DNA, 15 bp, Artificial Sequence
  Synthetic Polynucleotide
tgccctggatgggaa

SEQ ID NO: 9 - DNA, 21 bp, Artificial Sequence
  Synthetic Polynucleotide
tgcttgacacctggatgggaa

SEQ ID NO: 10 - DNA, 13 bp, Artificial Sequence
  Synthetic Polynucleotide
  misc_feature (13)..(13): Modified with /iSp18//iSp18//
tgctggatgggaa

SEQ ID NO: 11 - DNA, 15 bp, Artificial Sequence
  Synthetic Polynucleotide
  misc_feature (15)..(15): Modified with /iSp18//iSp18//
tgccctggatgggaa

SEQ ID NO: 12 - DNA, 21 bp, Artificial Sequence
  Synthetic Polynucleotide
  misc_feature (21)..(21): Modified with /iSp18//iSp18//
tgcttgacacctggatgggaa

SEQ ID NO: 13 - DNA, 15 bp, Artificial Sequence
  Synthetic Polynucleotide
  misc_feature (15)..(15): Modified with /iSp18//iSp18//
tcctgagcttgaagt

SEQ ID NO: 14 - DNA, 15 bp, Artificial Sequence
  Synthetic Polynucleotide
  misc_feature (15)..(15): Modified with /iSp18//iSp18//
ttctggcggggaagt

SEQ ID NO: 15 - DNA, 21 bp, Artificial Sequence
  Synthetic Polynucleotide
  misc_feature (21)..(21): Modified with /iSp18//iSp18//
ctcctattgggggtttcctat

SEQ ID NO: 16 - DNA, 26 bp, Artificial Sequence
  Synthetic Polynucleotide
  misc_feature (26)..(26): Modified with /iSp18//iSp18//
accccctctaccccctctacccctct

SEQ ID NO: 17 - DNA, 12 bp, Artificial Sequence
  Synthetic Polynucleotide
  misc_feature (12)..(12): Modified with /iSp18//iSp18//
cctggatgggaa

SEQ ID NO: 18 - DNA, 15 bp, Artificial Sequence
  Synthetic Polynucleotide
  misc_feature (15)..(15): Modified with /iSp18//iSp18//
ttctggcggggaagt

SEQ ID NO: 19 - DNA, 21 bp, Artificial Sequence
  Synthetic Polynucleotide
  misc_feature (21)..(21): Modified with /iSp18//iSp18//
ctcctattgggggtttcctat

SEQ ID NO: 20 - DNA, 26 bp, Artificial Sequence
  Synthetic Polynucleotide
  misc_feature (26)..(26): Modified with /iSp18//iSp18//
accccctctaccccctctacccctct

SEQ ID NO: 21 - DNA, 12 bp, Artificial Sequence
  Synthetic Polynucleotide
  misc_feature (12)..(12): Modified with /iSp18//iSp18//
cctggatgggaa

SEQ ID NO: 22 - DNA, 12 bp, Artificial Sequence
  Synthetic Polynucleotide
  misc_feature (1)..(4): Modified with a phosphorothioate bond
  misc_feature (12)..(12): Modified with /iSp18//iSp18//
cctggatgggaa

SEQ ID NO: 23 - DNA, 12 bp, Artificial Sequence
  Synthetic Polynucleotide
  misc_feature (8)..(11): Modified with a phosphorothioate bond
  misc_feature (12)..(12): Modified with /iSp18//iSp18//
cctggatgggaa

SEQ ID NO: 24 - DNA, 12 bp, Artificial Sequence
  Synthetic Polynucleotide
  misc_feature (1)..(4): Modified with a phosphorothioate bond
  misc_feature (8)..(11): Modified with a phosphorothioate bond
  misc_feature (12)..(12): Modified with /iSp18//iSp18//
cctggatgggaa

SEQ ID NO: 25 - DNA, 12 bp, Artificial Sequence
  Synthetic Polynucleotide
  misc_feature (1)..(1): Modified with /Chol/
  misc_feature (12)..(12): Modified with /iSp18//iSp18//
cctggatgggaa

SEQ ID NO: 26 - DNA, 12 bp, Artificial Sequence
  Synthetic Polynucleotide
  misc_feature (1)..(1): Modified with /Stryl/
  misc_feature (12)..(12): Modified with /iSp18//iSp18//
cctggatgggaa

SEQ ID NO: 27 - DNA, 12 bp, Artificial Sequence
  Synthetic Polynucleotide
  misc_feature (1)..(1): Modified with /Palm/
  misc_feature (12)..(12): Modified with /iSp18//iSp18//
cctggatgggaa

SEQ ID NO: 28 - DNA, 15 bp, Artificial Sequence
  Synthetic Polynucleotide
  misc_feature (1)..(15): Modified with a phosphorothioate bond
tcctggaggggttgt

SEQ ID NO: 29 - DNA, 18 bp, Artificial Sequence
  Synthetic Polynucleotide
  misc_feature (1)..(18): Modified with a phosphorothioate bond
tgctcctggaggggttgt

SEQ ID NO: 30 - DNA, 13 bp, Artificial Sequence
  Synthetic Polynucleotide
  misc_feature (1)..(13): Modified with a phosphorothioate bond
tgctggatgggaa

SEQ ID NO: 31 - DNA, 14 bp, Artificial Sequence
  Synthetic Polynucleotide
  misc_feature (1)..(14): Modified with a phosphorothioate bond
tgccctggatggga

SEQ ID NO: 32 - DNA, 21 bp, Artificial Sequence
  Synthetic Polynucleotide
  misc_feature (1)..(21): Modified with a phosphorothioate bond
tgcttgacacctggatgggaa

SEQ ID NO: 33 - DNA, 13 bp, Artificial Sequence
  Synthetic Polynucleotide
  misc_feature (1)..(13): Modified with a phosphorothioate bond
  misc_feature (13)..(13): Modified with /iSp18//iSp18//
tgctggatgggaa

SEQ ID NO: 34 - DNA, 15 bp, Artificial Sequence
  Synthetic Polynucleotide
  misc_feature (1)..(15): Modified with a phosphorothioate bond
  misc_feature (15)..(15): Modified with /iSp18//iSp18//
tgccctggatgggaa

SEQ ID NO: 35 - DNA, 21 bp, Artificial Sequence
  Synthetic Polynucleotide
  misc_feature (1)..(21): Modified with a phosphorothioate bond
  misc_feature (21)..(21): Modified with /iSp18//iSp18//
tgcttgacacctggatgggaa

SEQ ID NO: 36 - DNA, 15 bp, Artificial Sequence
  Synthetic Polynucleotide
  misc_feature (1)..(15): Modified with a phosphorothioate bond
  misc_feature (15)..(15): Modified with /iSp18//iSp18//
tcctgagcttgaagt

SEQ ID NO: 37 - DNA, 15 bp, Artificial Sequence
  Synthetic Polynucleotide
  misc_feature (1)..(15): Modified with a phosphorothioate bond
  misc_feature (15)..(15): Modified with /iSp18//iSp18//
ttctggcggggaagt

SEQ ID NO: 38 - DNA, 21 bp, Artificial Sequence
  Synthetic Polynucleotide
  misc_feature (1)..(21): Modified with a phosphorothioate bond
  misc_feature (21)..(21): Modified with /iSp18//iSp18//
ctcctattgggggtttcctat

SEQ ID NO: 39 - DNA, 26 bp, Artificial Sequence
  Synthetic Polynucleotide
  misc_feature (1)..(26): Modified with a phosphorothioate bond
  misc_feature (26)..(26): Modified with /iSp18//iSp18//
accccctctaccccctctacccctct

SEQ ID NO: 40 - DNA, 12 bp, Artificial Sequence
  Synthetic Polynucleotide
  misc_feature (1)..(12): Modified with a phosphorothioate bond
  misc_feature (12)..(12): Modified with /iSp18//iSp18//
cctggatgggaa

SEQ ID NO: 41 - DNA, 13 bp, Artificial Sequence
  Synthetic Polynucleotide
  misc_feature (1)..(1): Modified with /iSp18//iSp18//
  misc_feature (1)..(13): Modified with a phosphorothioate bond
tgctggatgggaa

SEQ ID NO: 42 - DNA, 15 bp, Artificial Sequence
  Synthetic Polynucleotide
  misc_feature (1)..(1): Modified with /iSp18//iSp18//
  misc_feature (1)..(15): Modified with a phosphorothioate bond
tgccctggatgggaa

SEQ ID NO: 43 - DNA, 21 bp, Artificial Sequence
  Synthetic Polynucleotide
  misc_feature (1)..(1): Modified with /iSp18//iSp18//
  misc_feature (1)..(21): Modified with a phosphorothioate bond
tgcttgacacctggatgggaa

SEQ ID NO: 44 - DNA, 15 bp, Artificial Sequence
  Synthetic Polynucleotide
  misc_feature (1)..(1): Modified with /iSp18//iSp18//
  misc_feature (1)..(15): Modified with a phosphorothioate bond
tcctgagcttgaagt

SEQ ID NO: 45 - DNA, 15 bp, Artificial Sequence
  Synthetic Polynucleotide
  misc_feature (1)..(1): Modified with /iSp18//iSp18//
  misc_feature (1)..(15): Modified with a phosphorothioate bond
ttctggcggggaagt

SEQ ID NO: 46 - DNA, 21 bp, Artificial Sequence
  Synthetic Polynucleotide
  misc_feature (1)..(1): Modified with /iSp18//iSp18//
  misc_feature (1)..(21): Modified with a phosphorothioate bond
ctcctattgggggtttcctat

SEQ ID NO: 47 - DNA, 26 bp, Artificial Sequence
  Synthetic Polynucleotide
  misc_feature (1)..(1): Modified with /iSp18//iSp18//
  misc_feature (1)..(26): Modified with a phosphorothioate bond
accccctctaccccctctacccctct

SEQ ID NO: 48 - DNA, 12 bp, Artificial Sequence
  Synthetic Polynucleotide
  misc_feature (1)..(1): Modified with /iSp18//iSp18//
  misc_feature (1)..(12): Modified with a phosphorothioate bond
cctggatgggaa

SEQ ID NO: 49 - DNA, 24 bp, Artificial Sequence
  Synthetic Polynucleotide
ttagggttagggttagggttaggg

SEQ ID NO: 50 - DNA, 24 bp, Artificial Sequence
  Synthetic Polynucleotide
  misc_feature (1)..(24): Modified with a phosphorothioate bond
ttagggttagggttagggttaggg

SEQ ID NO: 51 - DNA, 24 bp, Artificial Sequence
  Synthetic Polynucleotide
  misc_feature (24)..(24): Modified with /iSp18//iSp18//
ttagggttagggttagggttaggg

SEQ ID NO: 52 - DNA, 24 bp, Artificial Sequence
  Synthetic Polynucleotide
  misc_feature (1)..(24): Modified with a phosphorothioate bond
  misc_feature (24)..(24): Modified with /iSp18//iSp18//
ttagggttagggttagggttaggg

SEQ ID NO: 53 - DNA, 24 bp, Artificial Sequence
  Synthetic Polynucleotide
  misc_feature (1)..(1): Modified with /iSp18//iSp18//
ttagggttagggttagggttaggg

SEQ ID NO: 54 - DNA, 24 bp, Artificial Sequence
  Synthetic Polynucleotide
  misc_feature (1)..(1): Modified with /iSp18//iSp18//
  misc_feature (1)..(24): Modified with a phosphorothioate bond
ttagggttagggttagggttaggg

SEQ ID NO: 55 - DNA, 18 bp, Artificial Sequence
  Synthetic Polynucleotide
ctatctgucgttctctgu

SEQ ID NO: 56 - DNA, 18 bp, Artificial Sequence
  Synthetic Polynucleotide
  misc_feature (1)..(18): Modified with a phosphorothioate bond
ctatctgucgttctctgu

SEQ ID NO: 57 - DNA, 18 bp, Artificial Sequence
  Synthetic Polynucleotide
  misc_feature (18)..(18): Modified with /iSp18//iSp18//
ctatctgucgttctctgu

SEQ ID NO: 58 - DNA, 18 bp, Artificial Sequence
  Synthetic Polynucleotide
  misc_feature (1)..(18): Modified with a phosphorothioate bond
  misc_feature (24)..(24): Modified with /iSp18//iSp18//
ctatctgucgttctctgu

SEQ ID NO: 59 - DNA, 18 bp, Artificial Sequence
  Synthetic Polynucleotide
  misc_feature (1)..(1): Modified with /iSp18//iSp18//
ctatctgucgttctctgu

SEQ ID NO: 60 - DNA, 18 bp, Artificial Sequence
  Synthetic Polynucleotide
  misc_feature (1)..(1): Modified with /iSp18//iSp18//
  misc_feature (1)..(18): Modified with a phosphorothioate bond
ctatctgucgttctctgu

SEQ ID NO: 61 - DNA, 24 bp, Artificial Sequence
  Synthetic Polynucleotide
  misc_feature (1)..(1): Modified with /iSp18//iSp18//
  misc_feature (1)..(24): Modified with a phosphorothioate bond
ttagggttagggttagggttaggg

SEQ ID NO: 62 - DNA, 13 bp, Artificial Sequence
  Synthetic Polynucleotide
  misc_feature (1)..(1): Modified with /iSp18//iSp18//
tgctggatgggaa

SEQ ID NO: 63 - DNA, 15 bp, Artificial Sequence
  Synthetic Polynucleotide
  misc_feature (1)..(1): Modified with /iSp18//iSp18//
tgccctggatgggaa

SEQ ID NO: 64 - DNA, 21 bp, Artificial Sequence
  Synthetic Polynucleotide
  misc_feature (1)..(1): Modified with /iSp18//iSp18//
tgcttgacacctggatgggaa

SEQ ID NO: 65 - DNA, 15 bp, Artificial Sequence
  Synthetic Polynucleotide
  misc_feature (1)..(1): Modified with /iSp18//iSp18//
tcctgagcttgaagt

SEQ ID NO: 66 - DNA, 15 bp, Artificial Sequence
  Synthetic Polynucleotide
  misc_feature (1)..(1): Modified with /iSp18//iSp18//
ttctggcggggaagt

SEQ ID NO: 67 - DNA, 21 bp, Artificial Sequence
  Synthetic Polynucleotide
  misc_feature (1)..(1): Modified with /iSp18//iSp18//
ctcctattgggggtttcctat

SEQ ID NO: 68 - DNA, 26 bp, Artificial Sequence
  Synthetic Polynucleotide
  misc_feature (1)..(1): Modified with /iSp18//iSp18//
accccctctaccccctctacccctct

SEQ ID NO: 69 - DNA, 12 bp, Artificial Sequence
  Synthetic Polynucleotide
  misc_feature (1)..(1): Modified with /iSp18//iSp18//
cctggatgggaa

SEQ ID NO: 70 - DNA, 12 bp, Artificial Sequence
  Synthetic Polynucleotide
  misc_feature (1)..(1): Modified with /iSp18//iSp18//
  misc_feature (1)..(4): Modified with a phosphorothioate bond
cctggatgggaa

SEQ ID NO: 71 - DNA, 12 bp, Artificial Sequence
  Synthetic Polynucleotide
  misc_feature (1)..(1): Modified with /iSp18//iSp18//
  misc_feature (8)..(11): Modified with a phosphorothioate bond
cctggatgggaa

SEQ ID NO: 72 - DNA, 12 bp, Artificial Sequence
  Synthetic Polynucleotide
  misc_feature (1)..(1): Modified with /iSp18//iSp18//
  misc_feature (1)..(4): Modified with a phosphorothioate bond
  misc_feature (8)..(11): Modified with a phosphorothioate bond
cctggatgggaa

SEQ ID NO: 73 - DNA, 12 bp, Artificial Sequence
  Synthetic Polynucleotide
  misc_feature (1)..(1): Modified with /iSp18//iSp18//
  misc_feature (12)..(12): Modified with /Chol/
cctggatgggaa

SEQ ID NO: 74 - DNA, 12 bp, Artificial Sequence
  Synthetic Polynucleotide
  misc_feature (1)..(1): Modified with /iSp18//iSp18//
  misc_feature (12)..(12): Modified with /Stryl/
cctggatgggaa

SEQ ID NO: 75 - DNA, 12 bp, Artificial Sequence
  Synthetic Polynucleotide
  misc_feature (1)..(1): Modified with /iSp18//iSp18//
  misc_feature (12)..(12): Modified with /Palm/
cctggatgggaa

SEQ ID NO: 76 - skipped

SEQ ID NO: 77 - DNA, 18 bp, Artificial Sequence
  Synthetic Polynucleotide
  misc_feature (1)..(1): Modified with /iSp18//iSp18/
  misc_feature (1)..(18): Modified with a phosphorothioate bond
ctatctgucgttctctgu

SEQ ID NO: 78 - RNA, 18 nt, Artificial Sequence
  Synthetic Polynucleotide
ccgucuguugugugacuc

SEQ ID NO: 79 - RNA, 20 nt, Artificial Sequence
  Synthetic Polynucleotide
gccaccgagccgaaggcacc

SEQ ID NO: 80 - RNA, 20 nt, Artificial Sequence
  Synthetic Polynucleotide
uauauauauauauauauaua

SEQ ID NO: 81 - RNA, 20 nt, Artificial Sequence
  Synthetic Polynucleotide
uuauuauuauuauuauuauu

SEQ ID NO: 82 - RNA, 20 nt, Artificial Sequence
  Synthetic Polynucleotide
uuuuauuuuauuuuauuuua

SEQ ID NO: 83 - RNA, 20 nt, Artificial Sequence
  Synthetic Polynucleotide
ugugugugugugugugugug

SEQ ID NO: 84 - RNA, 20 nt, Artificial Sequence
  Synthetic Polynucleotide
uuguuguuguuguuguuguu

SEQ ID NO: 85 - RNA, 20 nt, Artificial Sequence
  Synthetic Polynucleotide
uuuguuuguuuguuuguuug

SEQ ID NO: 86 - RNA, 24 nt, Artificial Sequence
  Synthetic Polynucleotide
uuauuuauuuauuuauuuauuuau

SEQ ID NO: 87 - RNA, 24 nt, Artificial Sequence
  Synthetic Polynucleotide
uuguuuguuuguuuguuuguuugu

SEQ ID NO: 88 - RNA, 20 nt, Artificial Sequence
  Synthetic Polynucleotide
gcccgucuguugugugacuc

SEQ ID NO: 89 - DNA, 20 bp, Artificial Sequence
  Synthetic Polynucleotide
ggtgcatcgatgcagggggg

SEQ ID NO: 90 - DNA, 22 bp, Artificial Sequence
  Synthetic Polynucleotide
tccatggacgttcctgagcgtt

SEQ ID NO: 91 - DNA, 22 bp, Artificial Sequence
  Synthetic Polynucleotide
tcgtcgttcgaacgacgttgat

SEQ ID NO: 92 - DNA, 22 bp, Artificial Sequence
  Synthetic Polynucleotide
tcgtcgacgatccgcgcgcgcg

SEQ ID NO: 93 - DNA, 20 bp, Artificial Sequence
  Synthetic Polynucleotide
ggggtcaacgttgagggggg

SEQ ID NO: 94 - DNA, 24 bp, Artificial Sequence
  Synthetic Polynucleotide
tcgtcgttttgtcgttttgtcgtt

SEQ ID NO: 95 - DNA, 22 bp, Artificial Sequence
  Synthetic Polynucleotide
tcgtcgttgtcgttttgtcgtt

SEQ ID NO: 96 - DNA, 20 bp, Artificial Sequence
  Synthetic Polynucleotide
gggggacgatcgtcgggggg

SEQ ID NO: 97 - DNA, 21 bp, Artificial Sequence
  Synthetic Polynucleotide
ggggacgacgtcgtggggggg

SEQ ID NO: 98 - DNA, 22 bp, Artificial Sequence
  Synthetic Polynucleotide
tcgtcgttttcggcgcgcgccg

(19) United States
(12) Patent Application Publication
(10) Pub. No.: US 2020/0297867 A1
(43) Pub. Date: Sep. 24, 2020

(54) SURFACE FUNCTIONALIZATION OF LIPOSOMES AND LIPOSOMAL SPHERICAL NUCLEIC ACIDS (SNAS)

(71) Applicant: EXICURE,INC., Skokie, IL (US)

(72) Inventors: Richard Kang, Wilmette, IL (US); Sagar Anantatmula, Chicago, IL (US); Rishika Agarwal, Chicago, IL (US); Merideth Burkhart, Chicago, IL (US); Bart Anderson, Morton Grove, IL (US)

(73) Assignee: Exicure, Inc., Suite 410, IL (US)

(21) Appl. No.: 16/074,504

(22) PCT Filed: Feb. 1, 2017
(86) PCT No.: PCT/US2017/016084

§ 371 Date: Aug. 1, 2018

Related U.S. Application Data
(60) Provisional application No. 62/289,818, filed on Feb. 1, 2016.
(60) Provisional application No. 62/290,665, filed on Feb. 3, 2016.

(51) Int. Cl. A61K 47/69; A61K 9/127; A61K 31/711

(52) U.S. Cl. CPC A61K 47/6911; B82Y 5/00; A61K 31/711; A61K 9/1271

(57) ABSTRACT

The invention relates to a method of synthesizing liposomes and liposomal spherical nucleic acids with hydrophobic molecules functionalized to the surface. The lipid particles contain one or more agents that elicit an immune response.

### RELATED APPLICATIONS

This application claims priority under 35 U.S.C. § 119(e) to U.S. Provisional Application Ser. No. 62/289,818, entitled “SURFACE FUNCTIONALIZATION OF LIPOSOMES AND LIPOSOMAL SPHERICAL NUCLEIC ACIDS (SNAs)” filed on Feb. 1, 2016, and U.S. Provisional Application Ser. No. 62/290,665, entitled “SURFACE FUNCTIONALIZATION OF LIPOSOMES AND LIPOSOMAL SPHERICAL NUCLEIC ACIDS (SNAs)” filed on Feb. 3, 2016, which are herein incorporated by reference in their entirety.

### FIELD OF INVENTION

The present invention generally relates to nanoparticles with hydrophobic molecules functionalized to the surface as well as methods and compositions thereof.

### BACKGROUND

Typically, liposomes are formed by extrusion. The addition of any hydrophobic molecules that are not water-soluble must be added to the lipid mixture while it is in organic solvent, before the extrusion process. After undergoing extrusion, or other method of liposome synthesis, these hydrophobic molecules will be distributed throughout the liposomal unilamellar membrane, likely buried within the bilayer. The distribution of hydrophilic regions of amphipathic molecules will be randomized in both the internal and external leaflet of the liposomal membrane. This poses a problem when hydrophobic molecules need to be conjugated to liposomal SNAs as their efficacy is affected by their presentation on the SNA surface.

### SUMMARY OF THE INVENTION

Methods for functionalizing a hydrophobic molecule to the surface of a liposome are provided herein. In some aspects the method involves mixing an aqueous solution of a liposome with a hydrophobic molecule in an organic solvent to produce a liposomal solution and removing the organic solvent from the liposomal solution to produce a nanostructure comprised of the liposome surface functionalized with the hydrophobic molecule.

In some embodiments, the liposome is mixed with the hydrophobic molecule in a ratio of 99:1 by percent weight. In other embodiments, the liposome is mixed with the hydrophobic molecule in a ratio of 97:3, 95:5, or 92:8 by percent weight.

Optionally, the nanostructure is mixed with an oligonucleotide. In some embodiments, 95% of the oligonucleotides are positioned on the surface of the nanostructure. In other embodiments, the oligonucleotide is a toll-like receptor 9 (TLR9) agonist. In some embodiments, the TLR9 receptor agonist is a CpG oligonucleotide. In other embodiments, the oligonucleotide has at least one phosphorothioate internucleotide linkage. In some embodiments, the oligonucleotide has four CpG motifs.

In some embodiments, the liposome is a neutral lipid, a zwitterionic lipid, a cationic lipid, and an anionic lipid. In other embodiments, the lipid is 1,2-dioleoyl-sn-glycero-3-phosphocholine (DOPC).

In some embodiments, the organic solvent is dimethyl sulfoxide (DMSO). In other embodiments, the hydrophobic molecule is dissolved in the DMSO at about 1 mg/mL. In yet other embodiments, the DMSO is removed from the liposomal solution by diafiltration using tangential-flow filtration (TFF).

In some embodiments, the liposome is a spherical nucleic acid (SNA).

In yet other embodiments, the hydrophobic molecule is monophosphoryl lipid A (MPLA) or glycopyranoside lipid A (GLA).

In some embodiments, the lipid concentration is determined by a phospholipid assay.

The invention in some aspects is a nanostructure having a liposome with at least 40, 40-60, 45-55, and 50 hydrophobic molecules functionalized on the surface of the liposome.

In some embodiments, at least 60%, at least 70%, at least 80%, at least 90%, and 100% of the hydrophobic molecules in the nanostructure are positioned on the surface of the liposome.

Optionally, the liposome has an oligonucleotide shell on the surface of the liposome. In some embodiments, at least 95% of the oligonucleotides in the oligonucleotide shell are attached to the lipid nanoparticle through a lipid anchor group.

In some embodiments, the oligonucleotides are toll-like receptor 9 (TLR9) agonists. In some embodiments, the TLR9 receptor agonist is a CpG oligonucleotide. In some embodiments, the oligonucleotides have at least two CpG motifs or four CpG motifs.

In yet other embodiments, the oligonucleotides have at least one phosphorothioate internucleotide linkage. In some embodiments, the oligonucleotides are comprised of a 3′ cholesterol lipid anchor group. In other embodiments, the oligonucleotides have a spacer between the oligonucleotide and the 3′ cholesterol lipid anchor group. In some embodiments, the spacer is hexa(ethylene glycol). In other embodiments, the oligonucleotides of the nanostructure described herein have a sequence of SEQ ID NO: 1.

In some embodiments, the oligonucleotides are toll-like receptor 7/8 (TLR7/8) agonists. In other embodiments, the oligonucleotides have an RNA sequence composed of a phosphorothioate backbone and six-repeat UUG sequence.

In some embodiments, the oligonucleotides have a 5′ cholesterol lipid anchor group. In yet other embodiments, the oligonucleotides have a sequence of SEQ ID NO: 2.

In some embodiments, the oligonucleotides are structurally identical oligonucleotides, including at least two structurally different oligonucleotides, and 2-10 different nucleotide sequences.

In some embodiments, the oligonucleotide shell has a density of 5-1,000, 100-1,000, or 500-1,000 oligonucleotides per nanostructure.

In yet other embodiments, the oligonucleotides have 5′-termini or a 3′-termini exposed to the outside surface of the nanostructure.

In some embodiments, the liposome of the nanostructure described herein has a lipid selected from the group consisting of a neutral lipid, a zwitterionic lipid, a cationic lipid, and an anionic lipid. In some embodiments, the lipid is 1,2-dioleoyl-sn-glycero-3-phosphocholine (DOPC).

In some embodiments, the liposome and the hydrophobic molecule are in a ratio of 99:1, 97:3, 95:5, or 92:8 by percent weight.

In some embodiments, the hydrophobic molecule is MPLA or GLA.

In some aspects, a method for treating a disease or disorder in a subject is provided herein. In some embodiments, the method for treating a disease or disorder in a subject comprises administering to the subject any of the nanostructures described herein to elicit an immune response and treat the disease or disorder.

In some embodiments, the disease or disorder is cancer. In some embodiments, the disease or disorder is asthma. In some embodiments, the disease or disorder is infection. In some embodiments, the disease or disorder is allergy.

In some embodiments, the subject is a mammal. In some embodiments, the subject is a human.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. The details of one or more embodiments of the invention are set forth in the accompanying Detailed Description, Examples, Claims, and Figures. Other features, objects, and advantages of the invention will be apparent from the description and from the claims.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A and 1B. Comparison of TLR 4 stimulation of cell lines using liposomes co-extruded with MPLA/GLA versus liposomes surface functionalized with MPLA/GLA after extrusion. FIG. 1A depicts the distribution of hydrophilic regions of amphipathic molecules will be randomized in both the internal and external leaflet of the liposomal membrane and that the method described herein functionalizes the molecules (e.g. MPLA/GLA) to the surface. Both types of liposomes were synthesized with 1%, 3%, 5%, or 8% MPLA/GLA (wt/wt). Liposomes were added to TLR blue cell lines and activation of TLR4 was assayed (FIG. 1B).

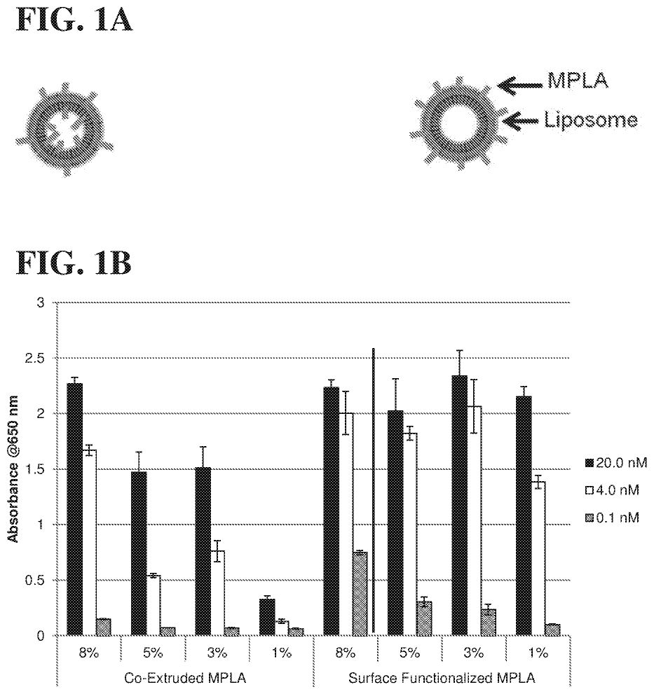

FIG. 2. Cytokine dose curve profile of PBMCs exposed to L-GLA (50 GLA/Liposome). All PBMCs were treated with a maximum concentration of 1 μM L-GLA (50 μM GLA) with a 1:2 serial dilution (8 total dilutions) and cytokine concentration was measured in pg/mL. Positive slope indicates dose-dependent response.

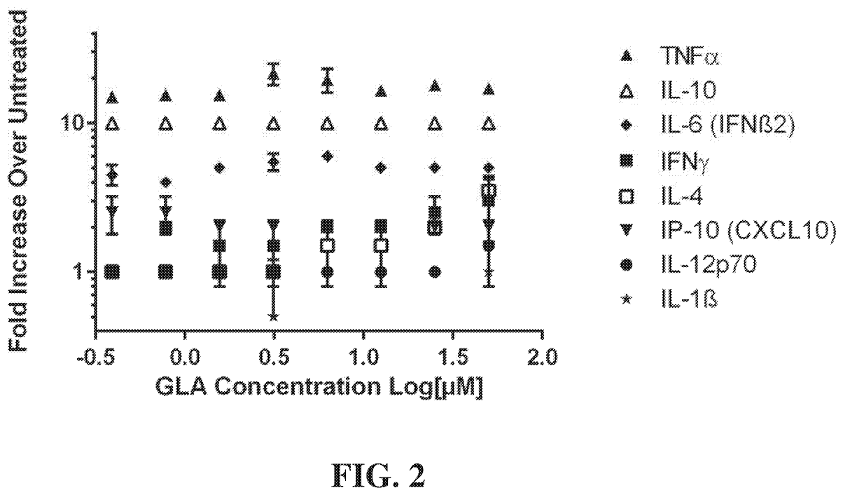

FIGS. 3A and 3B. Cytokine fold increase (compound vs. untreated) as a result of increasing GLA concentration when PBMCs are exposed to Multi-ligand SNAs (containing oligo 6589 (FIG. 3A) or oligo 7046 (FIG. 3B) and a variable dose of GLA). Cytokine analysis was performed in PBMCs tested against two sets of multi-ligand SNAs. In both cases, PBMCs were treated with L-GLA functionalized with oligo 6589 or oligo 7046 (see Multi-ligand Spherical Nucleic Acid Structure for compound concentrations tested). Cytokine concentration was measured in pg/mL. Positive slope indicates dose-dependent response.

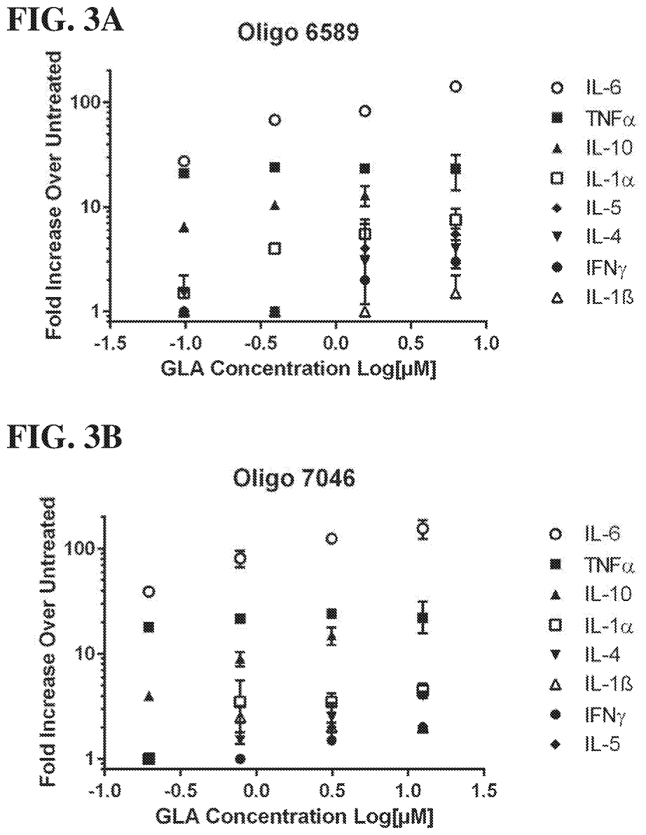

FIG. 4. L-GLA activates HEK hTLR4 post-sterile filtration through a 0.22 μM PES sterile syringe filter, but GLA alone does not. HEK hTLR4 cells were treated with 15 μM of GLA (for each compound tested) with a 1:3 serial dilution for 7 dilutions.

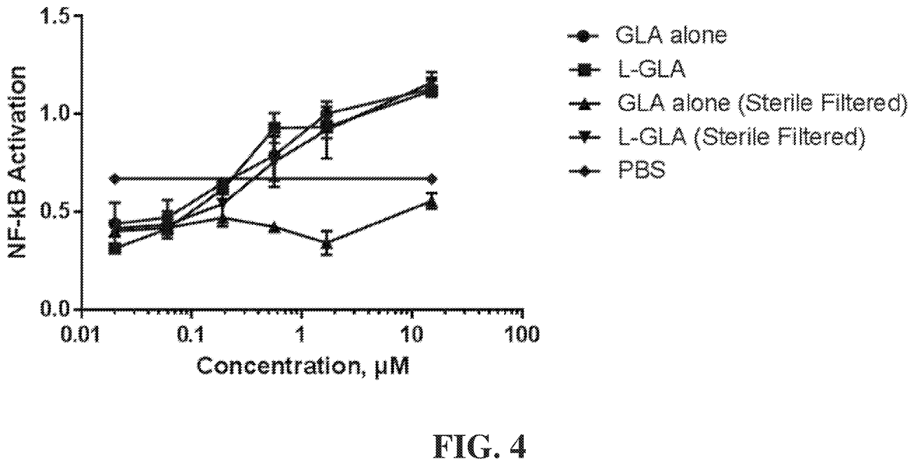

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The invention herein, in some aspects, describes a method of selectively functionalizing lipophilic and/or hydrophobic molecules to the surface of liposomes and liposomal spherical nucleic acids (SNAs). SNAs are a class of well-defined macromolecules, formed by organizing nucleic acids radially around an inorganic metallic nanoparticle core (Mirkin et al. (1996) Nature 382(6592):607-9). These structures exhibit the ability to enter cells without the need for auxiliary delivery vehicles or transfection reagents, without wishing to be bound by theory, by engaging class A scavenger receptors (SR-A) and lipid rafts (Patel et al. (2010) Bioconjug Chem 21(12):2250-6). Once inside the cell, the nucleic acid components of traditional SNAs resist nuclease degradation, leading to longer intracellular lifetimes.

In order to surface functionalize a hydrophobic molecule to the outer surface of a liposome or liposomal SNA, the liposomes or liposomal SNAs are externally functionalized with the hydrophobic molecule in an aqueous solution. The specific activity of the hydrophobic molecules molecule is retained after its functionalization to the surface of the liposome or liposomal SNA. In some aspects, the method described herein allows co-loading other molecules, including nucleic acids (e.g., oligonucleotides) for improved activity of the nanostructures. In some aspects, the nanostructures of the invention are useful for eliciting an immune or a cytokine response. The nanostructures allow co-loading of one or more agonists (e.g., TLR agonists) for improved cytokine response across multiple cytokines.

The nanostructures of the invention are typically composed of lipid nanoparticles having a lipophilic and/or hydrophobic molecule incorporated therein and, optionally, a shell of oligonucleotides, which is formed by arranging oligonucleotides such that they point radially outwards from the core. A hydrophobic or lipid anchor group (e.g., cholesterol) attached to either the 5′- or 3′-end of the oligonucleotide, depending on whether the oligonucleotides are arranged with the 5′- or 3′-end facing outward from the core preferably is used to embed the oligonucleotides in the lipid nanoparticle. The lipid anchor group acts to drive insertion into the lipid nanoparticle and to anchor the oligonucleotides to the lipids.

Incorporation of the lipophilic and/or hydrophobic molecules in the liposome or liposomal SNA construct confers unique properties on the structure including but not limited to enhanced bioavailability, enhanced targeting, enhanced drug product efficacy, enhanced in vivo pharmacodynamics and pharmacokinetic properties. Importantly, these nanostructures contain surface-functionalized lipophilic and/or hydrophobic molecules to elicit a cytokine response across multiple cytokines.

Achieving selective surface functionalization of lipophilic and/or hydrophobic molecules to elicit a cytokine response across multiple cytokines is a significant challenge. Typically, liposomes are formed by extrusion. The addition of any hydrophobic molecules that are not water-soluble must be added to the lipid mixture while it is in organic solvent, before the extrusion process. After extrusion or other methods of liposome synthesis known in the art, these hydrophobic molecules will be distributed throughout the liposomal unilamellar membrane, rather than localized at the surface. In contrast, the methods of the invention achieve the synthesis of liposomal SNAs with externally functionalized hydrophobic molecules in an aqueous solution such that the hydrophobic molecules can retain efficient activity. For instance, hydrophobic molecules that are TLR stimulating molecules and are formulated according to the methods of the invention provide an enhanced immune response across multiple cytokines relative the same molecules formulated according to the methods disclosed in the prior art.

Thus, the invention involves methods for functionalizing a hydrophobic molecule to the surface of a liposome by mixing an aqueous solution of a liposome with a hydrophobic molecule in an organic solvent to produce a liposomal solution and removing the organic solvent from the liposomal solution to produce a nanostructure comprised of the liposome surface functionalized with the hydrophobic molecule.

A liposome is a self-closed vesicular structure of various sizes and structures, where one or several membranes (e.g., lipid bilayer) encapsulate an internal compartment. Most typically liposome membranes are formed from lipid bilayers membranes, where the hydrophilic head groups are oriented towards the aqueous environment and the lipid chains are embedded in the lipophilic core. Liposomes can be formed as well from other amphiphilic monomeric and polymeric molecules, such as polymers, like block copolymers, or polypeptides. Liposomes may be characterized according to the membrane type and size. Small unilamellar vesicles (SUVs) have a single membrane and typically range between 0.02 and 0.05 pm in diameter; large unilamellar vesicles (LUVS) are typically larger than 0.05 pm. Oligolamellar large vesicles and multilamellar vesicles have multiple, usually concentric, membrane layers and are typically larger than 0.1 pm. Liposomes with several nonconcentric membranes, i.e., several smaller vesicles contained within a larger vesicle, are termed multivesicular vesicles.

The liposome may contain any of a number or types of lipids, including amphipathic, neutral, cationic, zwitterionic or anionic lipids. Such lipids can be used alone or in combination. Other lipids may be included in the lipid nanoparticle for a variety of purposes, such as to prevent lipid oxidation or to attach ligands onto lipid nanoparticle surface. Additional components that may be present in a lipid nanoparticle include bilayer stabilizing components such as polyamide oligomers, peptides, proteins, detergents, lipid-derivatives, such as PEG coupled to phosphatidylethanolamine and PEG conjugated to ceramides. The lipid nanoparticles may also include one or more of a second amino lipid or cationic lipid, a neutral lipid, a sterol, and a lipid selected to reduce aggregation of lipid particles during formation, which may result from steric stabilization of particles which prevents charge-induced aggregation during formation. As used herein, the term “amino lipid” is meant to include those lipids having one or two fatty acid or fatty alkyl chains and an amino head group (including an alkylamino or dialkylamino group) that may be protonated to form a cationic lipid at physiological pH.

In some embodiments, the lipid particles or liposomes have small sizes which may comprise a diameter from about 1 nm to about 250 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 1 nm to about 100 nm, from about 1 nm to about 110 nm, from about 1 nm to about 120 nm, from about 1 nm to about 130 nm, from about 1 nm to about 140 nm, from about 1 nm to about 150 nm, from about 1 nm to about 160 nm, from about 1 nm to about 170, from about 1 nm to about 180 nm, from about 1 nm to about 190 nm, from about 1 nm to about 200 nm, from about 1 nm to about 210 nm, from about 1 nm to about 220 nm, from about 1 nm to about 230 nm, from about 1 nm to about 240 nm, from about 1 nm to about 250 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, from about 5 nm to about 100 nm, from about 5 nm to about 110 nm, from about 5 nm to about 120 nm, from about 5 nm to about 130 nm, from about 5 nm to about 140 nm, from about 5 nm to about 150 nm, from about 5 nm to about 160 nm, from about 5 nm to about 170, from about 5 nm to about 180 nm, from about 5 nm to about 190 nm, from about 5 nm to about 200 nm, from about 5 nm to about 210 nm, from about 5 nm to about 220 nm, from about 5 nm to about 230 nm, from about 5 nm to about 240 nm, from about 5 nm to about 250 nm, about 10 to about 50 nm, from about 20 to about 50 nm, from about 20 to about 60 nm, from about 20 to about 70 nm, from about 20 to about 80 nm, from about 20 to about 90 nm, from about 20 to about 100 nm, from about 20 to about 150 nm, from about 20 to about 200 nm, from about 20 to about 250 nm, from about 30 to about 50 nm, from about 30 to about 60 nm, from about 30 to about 70 nm, from about 30 to about 80 nm, from about 30 to about 90 nm, from about 30 to about 100 nm, from about 30 to about 150 nm, from about 30 to about 300 nm, from about 30 to about 250 nm, from about 40 to about 50 nm, from about 40 to about 60 nm, from about 40 to about 70 nm, from about 40 to about 80 nm, from about 40 to about 90 nm, from about 40 to about 100 nm, from about 40 to about 150 nm, from about 40 to about 400 nm, from about 40 to about 250 nm, from about 50 to about 70 nm, from about 50 to about 80 nm, from about 50 to about 90 nm, from about 50 to about 100 nm, from about 50 to about 150 nm, from about 50 to about 500 nm, from about 50 to about 250 nm, from about 60 to about 70 nm, from about 60 to about 80 nm from about 60 to about 90 nm, from about 60 to about 100 nm, from about 60 to about 150 nm, from about 60 to about 600 nm, from about 60 to about 250 nm, from about 70 to about 90 nm, from about 70 to about 100 nm, from about 70 to about 150 nm, from about 70 to about 700 nm, and/or from about 70 to about 250 nm.

The nanostructure of the invention includes a core. The core may be a hollow core, which has at least some space in the center region of a shell material. Hollow cores include liposomal cores.

A liposomal core as used herein refers to a centrally located core compartment formed by a component of the lipids or phospholipids that form a lipid bilayer. The lipid bilayer is composed of two layers of lipid molecules. Each lipid molecule in a layer is oriented substantially parallel to adjacent lipid bilayers, and two layers that form a bilayer have the polar ends of their molecules exposed to the aqueous phase and the non-polar ends adjacent to each other. The central aqueous region of the liposomal core may be empty or filled fully or partially with water, an aqueous emulsion, oligonucleotides, or other therapeutic or diagnostic agent.

The liposomal core can be constructed from one or more lipids known to those in the art including but not limited to: sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and their derivatives.

“Lipid” refers to its conventional sense as a generic term encompassing fats, lipids, alcohol-ether-soluble constituents of protoplasm, which are insoluble in water.

Lipids usually consist of a hydrophilic and a hydrophobic moiety. In water lipids can self organize to form bilayers membranes, where the hydrophilic moieties (head groups) are oriented towards the aqueous phase, and the lipophilic moieties (acyl chains) are embedded in the bilayers core. Lipids can comprise as well two hydrophilic moieties (bola amphiphiles). In that case, membranes may be formed from a single lipid layer, and not a bilayer. Typical examples for lipids in the current context are fats, fatty oils, essential oils, waxes, steroid, sterols, phospholipids, glycolipids, sulpholipids, aminolipids, chromolipids, and fatty acids. The term encompasses both naturally occurring and synthetic lipids. Preferred lipids in connection with the present invention are: steroids and sterol, particularly cholesterol, phospholipids, including phosphatidyl, phosphatidylcholines and phosphatidylethanolamines and sphingomyelins. Where there are fatty acids, they could be about 12-24 carbon chains in length, containing up to 6 double bonds. The fatty acids are linked to the backbone, which may be derived from glycerol. The fatty acids within one lipid can be different (asymmetric), or there may be only 1 fatty acid chain present, e.g. lysolecithins. Mixed formulations are also possible, particularly when the non-cationic lipids are derived from natural sources, such as lecithins (phosphatidylcholines) purified from egg yolk, bovine heart, brain, liver or soybean.

In some embodiments, the lipid nanoparticle includes a neutral lipid. Non-limiting examples of a neutral lipid include 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), and 1,2-dihexadecanoyl-sn-glycero-3-phosphoethanolamine (DPPE), any related phosphatidylcholine or neutral lipids available from commercial vendors or known to one of ordinary skill in the art.

In some embodiments, the lipid nanoparticle includes a cationic lipid. Non-limiting examples of a cationic lipid include N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)proply)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.C1), 1,2-Dilinoleoyl-3-trimethylaminopropane chloride salt (DLin-TAP.C1), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro- -3aH-cyclopenta[d][1,3]dioxol-5-amine, (6Z,9Z,28Z,31Z)-heptatriaconta-6,9,28 ,31-tetraen- 19-yl-4-(dimethylamino)butanoate, or a mixture thereof, cationic lipids available from commercial vendors or known to one of ordinary skill in the art.

In some embodiments, other cationic lipids, which carry a net positive charge at about physiological pH, in addition to those specifically described above, are also included in the lipid nanoparticle. Non-limiting examples of cationic lipids include N,N-dioley-N,N-dimethylammonium chloride (“DODAC”); N-(2,3-dioleyloxy)propyl-N,N-N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 1,2-Dioleyloxy-3-trimethylaminopropane chloride salt (“DOTAP.Cl”); 3.beta.-(N-(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethyl- ammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), N,N-dimethyl-2,3-dioleyloxy)propylamine (“DODMA”), N-(1,2-dimyristyloxyprop-3-yl)-N,N-dimethyl-N-hydroxyethyl ammonium bromide (“DMRIE”), and 1,2-dioleoyl-sn-glycero-3-phosphocholine (“DOPC”).

In some embodiments, the lipid is a zwitterionic lipid. Non-limiting examples of zwitterionic lipids include 3-[(3-Cholamidopropyl)dimethylammonio]-1-propanesulfonate phosphatidyl choline (CHAPS), 3-[(3-Cholamidopropyl)dimethylammonio]-2-hydroxy-l-propanesulfonate (CHAPSO), N,N-dimethyldodecylamine N-oxide (LDAO), phosphatidyl ethanolamine, sphingomyelin, and other zwitterionic lipids known to one of ordinary skill in the art.

In some embodiments, the lipid is an anionic lipid. Non-limiting examples of anionic lipids include phosphatidylglycerol, phosphatidylserine, phosphatidic acid, phosphatidylinositol, P-glycerol, P-inositol, cardiolipin, and other anionic lipids known to one of ordinary skill in the art.

“Amphipathic lipids” refer to any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, dilinoleylphosphatidylcholine, monophosphoryl lipid A (MPLA), or glycopyranoside lipid A (GLA).

In some embodiments, the lipid concentration is determined by any technique known in the art. A non-limiting example of a technique to measure lipid concentration is a phospholipid assay.

The liposome is formulated in an aqueous solution. An aqueous solution is a water-based solution and includes aqueous solutions known to one of ordinary skill in the art.

The liposomal aqueous based solution is mixed with an organic solvent. An organic solvent is a carbon based solution that is capable of dissolving another substance and is miscible in an aqueous solution. Non-limiting examples of organic solvents include benzene, toluene, xylene, tetrahydrofurane, methyltetrahydrofurane, N,N-dimethylformamide, acetone, acetonitrile, anisole, dichloromethane, dimethylsulfoxide (DMSO), chlorobenzene, 1,2-dichlorobenzene and mixtures thereof.

In some embodiments, the hydrophobic molecule is dissolved in the organic solvent (e.g., DMSO) at a concentration of at least or about 1000 mg/mL, at least or about 500 mg/mL, at least or about 250 mg/mL, at least or about 200 mg/mL, at least or about 150 mg/mL, at least or about 100 mg/mL, at least or about 75 mg/mL, at least or about 50 mg/mL, at least or about 25 mg/mL, at least or about 20 mg/mL, at least or about 15 mg/mL, at least or about 10 mg/mL, at least or about 5 mg/mL, at least or about 1 mg/mL, at least or about 0.5 mg/mL, or at least or about 0.1 mg/mL.

The organic solvent is then removed from the liposomal solution using any technique known in the art. One example of a useful technique is diafiltration using tangential-flow filtration (TFF).

In some embodiments, the liposome is mixed with the hydrophobic molecule in a ratio of 50:50, 55:45, 60:40, 65:35, 70:30, 75:25, 80:20, 85:15, or 90:10 by percent weight. In other embodiments, the liposome is mixed with the hydrophobic molecule in a ratio of 97:3, 95:5, or 92:8 by percent weight. In certain embodiments, the liposome is mixed in a ratio of 99:1 by percent weight.

The methods are performed in order to position the hydrophobic molecule on the surface of the liposome. The resultant nanostructure has, in some embodiments at least 10, at least 20, at least 25, at least 30, at least 35, at least 40, at least 45, at least 50, at least 55, at least 60 hydrophobic molecules functionalized on the surface of the liposome. In other embodiments 10-20, 10-40, 10-60, 20-30, 20-40, 20-50, 20-60, 30-40, 30-50, 30-60, 30-70, 40-50, 40-60, 40-70, 40-80, 45-55, 50-60, 50-65, 50-70, 60-70 and 60-80 hydrophobic molecules are functionalized on the surface of the liposome.

Additionally the methods produce a nanostructure where a significant amount of the hydrophobic molecule incorporated therein is found on the surface as opposed to within the nanostructure. In some embodiments, at least 50%, at least 52%, at least 54%, at least 56%, at least 58%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% of the hydrophobic molecules in the nanostructure are positioned on the surface of the liposome.

In some embodiments at least 60%, 70%, 80%, 90%, 95%, 96%, 97%, 98%, 99% or 100% of the available surface area of the exterior surface of the core includes a hydrophobic molecule.

A hydrophobic molecule, as used herein, is a nonpolar molecule that repels water molecules and is used interchangeably with the term lipophilic molecule. In some embodiments the hydrophobic molecule may be selected from a TLR agonist, a TLR antagonist, an antigen, an adjuvant, a targeting molecule, or other pharmaceutical compound, such as an anti-cancer agent.

In some preferred embodiments, the hydrophobic molecule is a TLR agonist such as monophosphoryl lipid A (MPLA), also referred to as glycopyranoside lipid A (GLA). MPLA is a non-toxic derivative of lipopolysaccharide (LPS) originating from a strain of Salmonella. It essentially acts through Toll-like receptor 4 (TLR4). MPLA activates TLR4 but does not activate TLR2, even at high concentrations. MPLA and deacylated 3-O MPLA (or 3D-MPLA) both have a sugar backbone on which long chain fatty acids are attached and are highly hydrophobic molecules. MPLA has a structure:

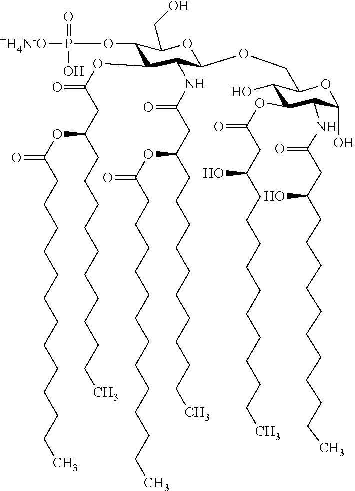

The hydrophobic molecule may also be a hydrophobic antigen or adjuvant. For instance, the antigen may be any antigen with amphipathic or hydrophobic groups, or rendered to have a hydrophobic region by rDNA expression and produced by cells or chemically synthesized. A hydrophobic adjuvant may be any adjuvant with amphipathic or hydrophobic groups such as those obtained from Quillaja saponaria Molina. In some embodiments the hydrophobic molecule is a TLR agonist or antagonist. A TLR agonist, as used herein is a molecule that interacts with and stimulates the activity of a TLR. A TLR antagonist, as used herein, is a molecule that interacts with and modulates, i.e. reduces, the activity of a TLR.

Toll-like receptors (TLRs) are a family of highly conserved polypeptides that play a critical role in innate immunity in mammals. At least ten family members, designated TLR1-TLR10, have been identified. The cytoplasmic domains of the various TLRs are characterized by a Toll-interleukin 1 (IL-1) receptor (TIR) domain (Medzhitov R et al. (1998) Mol Cell 2:253-8). Recognition of microbial invasion by TLRs triggers activation of a signaling cascade that is evolutionarily conserved in Drosophila and mammals. The TIR domain-containing adaptor protein MyD88 has been reported to associate with TLRs and to recruit IL-1 receptor-associated kinase (IRAK) and tumor necrosis factor (TNF) receptor-associated factor 6 (TRAF6) to the TLRs. The MyD88-dependent signaling pathway is believed to lead to activation of NF-κB transcription factors and c-Jun NH2 terminal kinase (Jnk) mitogen-activated protein kinases (MAPKs), critical steps in immune activation and production of inflammatory cytokines. For a review, see Aderem A et al. (2000) Nature 406:782-87. TLRs may be stimulated or inhibited with TLR agonists or antagonists, respectively.

A TLR4-mediated immune response is a response associated with TLR4 signaling. TLR4-mediated immune response is generally characterized by the induction of inflammatory cytokines, such as interleukin (IL)-6 and type 1 IFN. Activation of TLR4 promotes the NF-kappa-B-dependent production of CXCL1, CXCL2 and CCL9 cytokines, via MYD88 signaling pathway, and CCLS cytokine, via TICAM1 signaling pathway, as well as IL1B secretion. TLR4 is unique among the TLR family in that downstream signaling occurs via both the MyD88- and TRIF-dependent pathways. Collectively, these pathways stimulate dendritic cell maturation, antigen processing/presentation, T cell priming, and the production of cytokines (e.g., IL-12, IFNα/β, and TNFα) (see, e.g., Iwasaki et al., Na. Immunol. 5:987 (2004)).

A TLR7-mediated immune response is a response associated with TLR7 signaling. TLR7-mediated immune response is generally characterized by the induction of IFN-α and IFN-inducible cytokines such as IP-10 and I-TAC. The levels of cytokines IL-1 α/β, IL-6, IL-8, MIP-1α/β and MIP-3α/β induced in a TLR7-mediated immune response are less than those induced in a TLR8-mediated immune response.

A TLR8-mediated immune response is a response associated with TLR8 signaling. This response is further characterized by the induction of pro-inflammatory cytokines such as IFN-γ, IL-12p40/70, TNF-α, IL-1aα/β, IL-6, IL-8, MIP-1 α/β and MIP-3 α/β.

A TLR9-mediated immune response is a response associated with TLR9 signaling. This response is further characterized at least by the production/secretion of

IFN-γ and IL-12, albeit at levels lower than are achieved via a TLR8-mediated immune response.

As used herein, a “TLR4 agonist” collectively refers to a compound that is capable of increasing TLR4 signaling. TLR4 agonists include, without limitation, lipids, such as lipopolysaccharides (LPS), which is a component of Gram-negative bacteria, MPLA, synthetic forms of MPLA, and heat-killed Salmonella typhimurium, several viral proteins, polysaccharide, and a variety of endogenous proteins such as low-density lipoprotein, beta-defensins, and heat shock protein (see e.g. Brubaker et al. Annual Review of Immunology 33: 257-90). Drugs that serve as TLR4 agonists include, but are not limited to buprenorphine, carbamazepine, ethanol, fentanyl, levorphanol, methadone, morphine, oxcarbazepine, oxycodone, pethidine, glucuronoxylomannan from Cryptococcus, and morphine-3-glucuronide.

As used herein, a “TLR7/8 agonist” collectively refers to any nucleic acid that is capable of increasing TLR7 and/or TLR8 signaling (i.e., an agonist of TLR7 and/or TLR8). Some TLR7/8 ligands induce TLR7 signaling alone (e.g., TLR7 specific agonists), some induce TLR8 signaling alone (e.g., TLR8 specific agonists), and others induce both TLR7 and TLR8 signaling.

As used herein, the term “TLR9 agonist” refers to any agent that is capable of increasing TLR9 signaling (i.e., an agonist of TLR9). TLR9 agonists specifically include, without limitation, immunostimulatory nucleic acids, and in particular CpG immunostimulatory nucleic acids.

The hydrophobic containing nanostructure may also include one or more oligonucleotides incorporated therein and/or on the surface of the nanostructure. In some embodiments the oligonucleotides may form a shell around part or all of the nanostructure surface. An oligonucleotide, as used herein, refers to any nucleic acid containing molecule. The nucleic acid may be DNA, RNA, PNA, LNA, ENA or combinations or modifications thereof. It may also be single, double or triple stranded. A therapeutic oligonucleotide is an oligonucleotide that can function as a therapeutic and or diagnostic agent.

Therapeutic oligonucleotides include but are not limited to immunomodulatory oligonucleotides, inhibitory oligonucleotides, expression enhancing oligonucleotides and diagnostic oligonucleotides. In some embodiments the immunomodulatory oligonucleotide is a nucleic acid TLR agonist or antagonist, such as a TLR 4, 7, 8 or 9 agonist or antagonist.

An “immunostimulatory oligonucleotide” as used herein is any nucleic acid (DNA or RNA) containing an immunostimulatory motif or backbone that is capable of inducing an immune response. An induction of an immune response refers to any increase in number or activity of an immune cell, or an increase in expression or absolute levels of an immune factor, such as a cytokine. Immune cells include, but are not limited to, NK cells, CD4+ T lymphocytes, CD8+ T lymphocytes, B cells, dendritic cells, macrophage and other antigen-presenting cells. Cytokines include, but are not limited to, interleukins, TNF, IFN-α, β, and γ, Flt-ligand, and co-stimulatory molecules.

Immunostimulatory motifs include, but are not limited to CpG motifs.

The immunostimulatory oligonucleotides of the nanoscale construct are preferably in the range of 6 to 100 bases in length. However, nucleic acids of any size greater than 6 nucleotides (even many kb long) are capable of inducing an immune response according to the invention if sufficient immunostimulatory motifs are present.

Preferably the immunostimulatory nucleic acid is in the range of between 8 and 100 and in some embodiments between 8 and 50 or 8 and 30 nucleotides in size.

As used herein, the term “immunostimulatory CpG nucleic acids” or “immunostimulatory CpG oligonucleotides” or “CpG oligonucleotides” refers to any CpG-containing nucleic acid that is capable of activating an immune cell. At least the C of the CpG dinucleotide is typically, but not necessarily, unmethylated. Immunostimulatory CpG nucleic acids are described in a number of issued patents and published patent applications, including U.S. Pat. Nos. 6,194,388; 6,207,646; 6,218,371; 6,239,116; 6,339,068; 6,406,705; and 6,429,199.

In some embodiments, the CpG oligonucleotide has ten CpG motifs, nine CpG motifs, eight CpG motifs, seven CpG motifs, six CpG motifs, five CpG motifs, four CpG motifs, three CpG motifs, two CpG motifs, or one CpG motif.

In some embodiments, the immunostimulatory oligonucleotides have at least one phosphorothioate (PS) internucleotide linkage. In some embodiments, the immunostimulatory oligonucleotides have a modified backbone such as a PS backbone.

In other embodiments, the immunostimulatory oligonucleotides have at least one phosphodiester (PO) linkage. In some embodiments, the immunostimulatory oligonucleotides have a PO backbone. In yet other embodiments immunostimulatory oligonucleotides have a mixed PO and PS backbone.

A non-limiting set of immunostimulatory oligonucleotides includes:

dsRNA (TLR 3): poly(A:U) and poly(I:C)
ssRNA (TLR7/8):
(SEQ ID NO: 78)
CCGUCUGUUGUGUGACUC
(SEQ ID NO: 79)
GCCACCGAGCCGAAGGCACC
(SEQ ID NO: 80)
UAUAUAUAUAUAUAUAUAUA
(SEQ ID NO: 81)
UUAUUAUUAUUAUUAUUAUU
(SEQ ID NO: 82)
UUUUAUUUUAUUUUAUUUUA
(SEQ ID NO: 83)
UGUGUGUGUGUGUGUGUGUG
(SEQ ID NO: 84)
UUGUUGUUGUUGUUGUUGUU
(SEQ ID NO: 85)
UUUGUUUGUUUGUUUGUUUG
(SEQ ID NO: 86)
UUAUUUAUUUAUUUAUUUAUUUAU
(SEQ ID NO: 87)
UUGUUUGUUUGUUUGUUUGUUUGU
(SEQ ID NO: 88)
GCCCGUCUGUUGUGUGACUC
(SEQ ID NO: 3)
GUCCUUCAAGUCCUUCAA
DNA (TLR9):
(SEQ ID NO: 89)
GGTGCATCGATGCAGGGGGG
(SEQ ID NO: 90)
TCCATGGACGTTCCTGAGCGTT
(SEQ ID NO: 91)
TCGTCGTTCGAACGACGTTGAT
(SEQ ID NO: 92)
TCGTCGACGATCCGCGCGCGCG
(SEQ ID NO: 93)
GGGGTCAACGTTGAGGGGGG
(SEQ ID NO: 94)
TCGTCGTTTTGTCGTTTTGTCGTT
(SEQ ID NO: 95)
TCGTCGTTGTCGTTTTGTCGTT
(SEQ ID NO: 96)
GGGGGACGATCGTCGGGGGG
(SEQ ID NO: 97)
GGGGACGACGTCGTGGGGGGG
(SEQ ID NO: 98)
TCGTCGTTTTCGGCGCGCGCCG
(SEQ ID NO: 4)
TCGTCGTCGTTCGAACGACGTTGAT

As used herein, a “TLR7/8 antagonist” collectively refers to any nucleic acid that is capable of decreasing TLR7 and/or TLR8 signaling (i.e., an antagonist of TLR7 and/or TLR8) relative to a baseline level. Some TLR7/8 antagonists decrease TLR7 signaling alone (e.g., TLR7 specific antagonists), some decrease TLR8 signaling alone (e.g., TLR8 specific antagonists), and others decrease both TLR7 and TLR8 signaling.

As used herein, the term “TLR9 antagonist” refers to any agent that is capable of decreasing TLR9 signaling (i.e., an antagonist of TLR9).

In some embodiments antagonists of TLR 7, 8, or 9 include immunoregulatory nucleic acids. Immunoregulatory nucleic acids include but are not limited to nucleic acids falling within the following formulas: 5′RₙJGCN₂3′, wherein each R is a nucleotide, n is an integer from about 0 to 10, J is U or T, each N is a nucleotide, and z is an integer from about 1 to about 100. In some embodiments, n is 0 and z is from about 1 to about 50. In some embodiments N is 5′S₁S₂S₃S₄3′, wherein S₁, S₂, S₃, and S₄ are independently G, I, or 7-deaza-dG. In some embodiments the TLR7 TLR8 and/or TLR9 antagonist is selected from the group consisting of

(SEQ ID NO: 5)
TCCTGGAGGGGTTGT,
(SEQ ID NO: 6)
TGCTCCTGGAGGGGTTGT,
(SEQ ID NO: 7)
TGCTGGATGGGAA,
(SEQ ID NO: 8)
TGCCCTGGATGGGAA,
(SEQ ID NO: 9)
TGCTTGACACCTGGATGGGAA,
(SEQ ID NO: 10)
TGCTGGATGGGAA/iSp18//iSp18//,
(SEQ ID NO: 11)
TGCCCTGGATGGGAA/i5p18//i5p18//,
(SEQ ID NO: 12)
TGCTTGACACCTGGATGGGAA/iSp18//iSp18//,
(SEQ ID NO: 13)
TCCTGAGCTTGAAGT/i5p18//i5p18/,
(SEQ ID NO: 14)
TTCTGGCGGGGAAGT/i5p18//i5p18/,
(SEQ ID NO: 15)
CTCCTATTGGGGGTTTCCTAT/iSp18//iSp18/,
(SEQ ID NO: 16)
ACCCCCTCTACCCCCTCTACCCCTCT/i5p18//i5p18/,
(SEQ ID NO: 17)
CCTGGATGGGAA/i5p18//i5p18/,
(SEQ ID NO: 18)
TTCTGGCGGGGAAGT/i5p18//i5p18//,
(SEQ ID NO: 19)
CTCCTATTGGGGGTTTCCTAT/iSp18//iSp18//,
(SEQ ID NO: 20)
ACCCCCTCTACCCCCTCTACCCCTCT/iSp18//iSp18//,
(SEQ ID NO: 21)
CCTGGATGGGAA/iSp18//iSp18//,
(SEQ ID NO: 22)
C*C*T*GGATGGGAA/iSp18//iSp18//,
(SEQ ID NO: 23)
CCTGGATG*G*G*AA/iSp18//iSp18//,
(SEQ ID NO: 24)
C*C*T*GGATG*G*G*AA/iSp18//iSp18//,
(SEQ ID NO: 25)
/Chol/CCTGGATGGGAA/iSp18//iSp18//,
(SEQ ID NO: 26)
/Stryl/CCTGGATGGGAA/iSp18//iSp18//,
(SEQ ID NO: 27)
/Palm/CCTGGATGGGAA/iSp18//iSp18//,
(SEQ ID NO: 28)
T*C*C*T*G*G*A*G*G*G*G*T*T*G*T,
(SEQ ID NO: 29)
T*G*C*T*C*C*T*G*G*A*G*G*G*G*T*T*G*T,
(SEQ ID NO: 30)
T*G*C*T*G*G*A*T*G*G*G*A*A,
(SEQ ID NO: 31)
T*G*C*C*C*T*G*G*A*T*G*G*G**A,
(SEQ ID NO: 32)
T*G*C*T*T*G*A*C*A*C*C*T*G*G*A*T*G*G*G*A*A,
(SEQ ID NO: 33)
T*G*C*T*G*G*A*T*G*G*G*A*A*/i5p18//i5p18//,
(SEQ ID NO: 34)
T*G*C*C*C*T*G*G*A*T*G*G*G*A*A*/i5p18//i5p18//,
(SEQ ID NO: 35)
T*G*C*T*T*G*A*C*A*C*C*T*G*G*A*T*G*G*G*A*A*/i5p18//
i5p18//,
(SEQ ID NO: 36)
T*C*C*T*G*A*G*C*T*T*G*A*A*G*T*/i5p18//i5p18//,
(SEQ ID NO: 37)
T*T*C*T*G*G*C*G*G*G*G*A*A*G*T*/i5p18//i5p18//,
(SEQ ID NO: 38)
C*T*C*C*T*A*T*T*G*G*G*G*G*T*T*T*C*C*T*A*T*/i5p18//
i5p18//,
(SEQ ID NO: 39)
A*C*C*C*C*C*T*C*T*A*C*C*C*C*C*T*C*T*A*C*C*C*C*T*C*
T*/iSp18//iSp18//,
(SEQ ID NO: 40)
C*C*T*G*G*A*T*G*G*G*A*A*/i5p18//i5p18//,
(SEQ ID NO: 41)
/i5p18//i5p18/*T*G*C*T*G*G*A*T*G*G*G*A*A,
(SEQ ID NO: 42)
/i5p18//i5p18/*T*G*C*C*C*T*G*G*A*T*G*G*G*A*A,
(SEQ ID NO: 43)
/i5p18//i5p18/*T*G*C*T*T*G*A*C*A*C*C*T*G*G*A*T*G*G
*G*A*A,
(SEQ ID NO: 44)
/i5p18//i5p18/*T*C*C*T*G*A*G*C*T*T*G*A*A*G*T,
(SEQ ID NO: 45)
/i5p18//i5p18/*T*T*C*T*G*G*C*G*G*G*G*A*A*G*T,
(SEQ ID NO: 46)
/i5p18//i5p18/*C*T*C*C*T*A*T*T*G*G*G*G*G*T*T*T*C*C
*T*A*T,
(SEQ ID NO: 47)
/iSp18//iSp18/*A*C*C*C*C*C*T*C*T*A*C*C*C*C*C*T*C*
T*A*C*C*C*C*T*C*T,
(SEQ ID NO: 48)
/i5p18//i5p18/*C*C*T*G*G*A*T*G*G*G*A*A,
(SEQ ID NO: 49)
TTAGGGTTAGGGTTAGGGTTAGGG,
(SEQ ID NO: 50)
T*T*A*G*G*G*T*T*A*G*G*G*T*T*A*G*G*G*T*T*A*G*G*G,
(SEQ ID NO: 51)
TTAGGGTTAGGGTTAGGGTTAGGG/iSp18//iSp18//,
(SEQ ID NO: 52)
T*T*A*G*G*G*T*T*A*G*G*G*T*T*A*G*G*G*T*T*A*G*G*G*/
iSp18//iSp18//,
(SEQ ID NO: 53)
/iSp18//iSp18/TTAGGGTTAGGGTTAGGGTTAGGG,
(SEQ ID NO: 54)
/iSp18//iSp18/*T*T*A*G*G*G*T*T*A*G*G*G*T*T*A*G*G*
G*T*T*A*G*G*G,
(SEQ ID NO: 55)
CTATCTGUCGTTCTCTGU,
(SEQ ID NO: 56)
C*T*A*T*C*T*G*U*C*G*T*T*C*T*C*T*G*U,
(SEQ ID NO: 57)
CTATCTGUCGTTCTCTGU/iSp18//iSp18//,
(SEQ ID NO: 58)
C*T*A*T*C*T*G*U*C*G*T*T*C*T*C*T*G*U*/i5p18//
i5p18//,
(SEQ ID NO: 59)
/i5p18//i5p18/CTATCTGUCGTTCTCTGU,
(SEQ ID NO: 60)
/i5p18//i5p18/*C*T*A*T*C*T*G*U*C*G*T*T*C*T*C*T*G*
U,
(SEQ ID NO: 61)
/iSp18//iSp18/T*T*A*G*G*G*T*T*A*G*G*G*T*T*A*G*G*G*
T*T*A*G*G*G*,
(SEQ ID NO: 77)
/i5p18//i5p18/C*T*A*T*C*T*G*U*C*G*T*T*C*T*C*T*G*
U*,
(SEQ ID NO: 62)
/iSp18//iSp18/TGCTGGATGGGAA,
(SEQ ID NO: 63)
/i5p18//i5p18/TGCCCTGGATGGGAA,
(SEQ ID NO: 64)
/iSp18//iSp18/TGCTTGACACCTGGATGGGAA,
(SEQ ID NO: 65)
/iSp18//iSp18/TCCTGAGCTTGAAGT,
(SEQ ID NO: 66)
/i5p18//i5p18/TTCTGGCGGGGAAGT,
(SEQ ID NO: 67)
/iSp18//iSp18/CTCCTATTGGGGGTTTCCTAT,
(SEQ ID NO: 68)
/i5p18//i5p18/ACCCCCTCTACCCCCTCTACCCCTCT,
(SEQ ID NO: 69)
/i5p18//i5p18/CCTGGATGGGAA,
(SEQ ID NO: 70)
/i5p18//i5p18/C*C*T*GGATGGGAA,
(SEQ ID NO: 71)
/iSp18//iSp18/CCTGGATG*G*G*AA,
(SEQ ID NO: 72)
/iSp18//iSp18/C*C*T*GGATG*G*G*AA,
(SEQ ID NO: 73)
/iSp18//iSp18/CCTGGATGGGAA/Chol/,
(SEQ ID NO: 74)
/iSp18//iSp18/CCTGGATGGGAA/Stryl/,
(SEQ ID NO: 75)
/iSp18//iSp18/CCTGGATGGGAA/Palm/,
(SEQ ID NO: 1)
T*C*G*T*C*G*T*T*T*T*G*T*C*G*T*T*T*T*G*T*C*G*T*T/
iSp18//iSp18/Chol/,
(SEQ ID NO: 2)
/Chol//iSp18//iSp18/rU*rU*rG*rU*rU*rG*rU*rU*rG*rU*
rU*rG*rU*rU*rG*rU*rU*rG*rU*rU.

“/Stryl/” in the nucleotide sequences denotes a C16/C18 Stearyl group and “/Palm/” in the nucleotide denotes a Palmitoyl group.

In other embodiments the oligonucleotide is an inhibitory nucleic acid. The oligonucleotide that is an inhibitory nucleic acid may be, for instance, an siRNA or an antisense molecule that inhibits expression of a protein that will have a therapeutic effect. The inhibitory nucleic acids may be designed using routine methods in the art.

An inhibitory nucleic acid typically causes specific gene knockdown, while avoiding off-target effects. Various strategies for gene knockdown known in the art can be used to inhibit gene expression. For example, gene knockdown strategies may be used that make use of RNA interference (RNAi) and/or microRNA (miRNA) pathways including small interfering RNA (siRNA), short hairpin RNA (shRNA), double-stranded RNA (dsRNA), miRNAs, and other small interfering nucleic acid-based molecules known in the art. In one embodiment, vector-based RNAi modalities (e.g., shRNA expression constructs) are used to reduce expression of a gene in a cell. In some embodiments, therapeutic compositions of the invention comprise an isolated plasmid vector (e.g., any isolated plasmid vector known in the art or disclosed herein) that expresses a small interfering nucleic acid such as an shRNA. The isolated plasmid may comprise a specific promoter operably linked to a gene encoding the small interfering nucleic acid. In some cases, the isolated plasmid vector is packaged in a virus capable of infecting the individual. Exemplary viruses include adenovirus, retrovirus, lentivirus, adeno-associated virus, and others that are known in the art and disclosed herein.

A broad range of RNAi-based modalities could be employed to inhibit expression of a gene in a cell, such as siRNA-based oligonucleotides and/or altered siRNA-based oligonucleotides. Altered siRNA based oligonucleotides are those modified to alter potency, target affinity, safety profile and/or stability, for example, to render them resistant or partially resistant to intracellular degradation. Modifications, such as phosphorothioates, for example, can be made to oligonucleotides to increase resistance to nuclease degradation, binding affinity and/or uptake. In addition, hydrophobization and bioconjugation enhances siRNA delivery and targeting (De Paula et al., RNA. (2007) 13(4):431-56) and siRNAs with ribo-difluorotoluyl nucleotides maintain gene silencing activity (Xia et al., ASC Chem. (2006) Biol. 1(3):176-83). siRNAs with amide-linked oligoribonucleosides have been generated that are more resistant to S1 nuclease degradation than unmodified siRNAs (Iwase et al. Nucleic Acids Symp Ser (2006) 50: 175-176). In addition, modification of siRNAs at the 2′-sugar position and phosphodiester linkage confers improved serum stability without loss of efficacy (Choung et al., Biochem. Biophys. Res. Commun. (2006) 342(3):919-26).

Other molecules that can be used to inhibit expression of a gene include antisense nucleic acids (single or double stranded), ribozymes, peptides, DNAzymes, peptide nucleic acids (PNAs), triple helix forming oligonucleotides, antibodies, and aptamers and modified form(s) thereof directed to sequences in gene(s), RNA transcripts, or proteins. Antisense and ribozyme suppression strategies have led to the reversal of a tumor phenotype by reducing expression of a gene product or by cleaving a mutant transcript at the site of the mutation (Carter et al. J. Cancer. (1993) 67(5):869-76; Lange et al., Leukemia. (1993) 6(11):1786-94; Valera et al., J. Biol. Chem. (1994) 269(46):28543-6; Dosaka-Akita et al., Am. J. Clin. Pathol. (1994) 102(5):660-4; Feng et al., Cancer Res. (1995) 55(10):2024-8; Quattrone et al., Cancer Res. (1995) 55(1):90-5; Lewin et al., Nat Med. (1998) 4(8):967-71). Ribozymes have also been proposed as a means of both inhibiting gene expression of a mutant gene and of correcting the mutant by targeted trans-splicing (Sullenger et al., Nature (1994) 371(6498):619-22; Jones et al., Nat. Med. (1996) 2(6):643-8).

Triple helix approaches have also been investigated for sequence-specific gene suppression. Triple helix forming oligonucleotides have been found in some cases to bind in a sequence-specific manner (Postel et al., Proc. Natl. Acad. Sci. U.S.A. 88(18):8227-31, 1991; Duval-Valentin et al., Proc. Natl. Acad. Sci. U.S.A. 89(2):504-8, 1992; Hardenbol and Van Dyke Proc. Natl. Acad. Sci. U.S.A. 93(7):2811-6, 1996; Porumb et al., Cancer Res. 56(3):515-22, 1996). Similarly, peptide nucleic acids have been shown to inhibit gene expression (Hanvey et al., Antisense Res. Dev. 1(4):307-17, 1991; Knudsen and Nielson Nucleic Acids Res. 24(3):494-500, 1996; Taylor et al., Arch. Surg. 132(11):1177-83, 1997). Minor-groove binding polyamides can bind in a sequence-specific manner to DNA targets and hence may represent useful small molecules for suppression at the DNA level (Trauger et al., Chem. Biol. 3(5):369-77, 1996). In addition, suppression has been obtained by interference at the protein level using dominant negative mutant peptides and antibodies (Herskowitz Nature 329(6136):219-22, 1987; Rimsky et al., Nature 341(6241):453-6, 1989; Wright et al., Proc. Natl. Acad. Sci. U.S.A. 86(9):3199-203, 1989). The diverse array of suppression strategies that can be employed includes the use of DNA and/or RNA aptamers that can be selected to target a protein of interest.

Other inhibitor molecules that can be used include antisense nucleic acids (single or double stranded). Antisense nucleic acids include modified or unmodified RNA, DNA, or mixed polymer nucleic acids, and primarily function by specifically binding to matching sequences resulting in modulation of peptide synthesis (Wu-Pong, BioPharm (1994) 20-33). Antisense nucleic acid binds to target RNA by Watson Crick base-pairing and blocks gene expression by preventing ribosomal translation of the bound sequences either by steric blocking or by activating RNase H enzyme. Antisense molecules may also alter protein synthesis by interfering with RNA processing or transport from the nucleus into the cytoplasm (Mukhopadhyay et al., Crit. Rev. in Oncogenesis (1996) 7, 151-190).

As used herein, the term “antisense nucleic acid” describes a nucleic acid that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence.

An inhibitory nucleic acid useful in the invention will generally be designed to have partial or complete complementarity with one or more target genes. The target gene may be a gene derived from the cell, an endogenous gene, a transgene, or a gene of a pathogen which is present in the cell after infection thereof. Depending on the particular target gene, the nature of the inhibitory nucleic acid and the level of expression of inhibitory nucleic acid (e.g. depending on copy number, promoter strength) the procedure may provide partial or complete loss of function for the target gene. Quantitation of gene expression in a cell may show similar amounts of inhibition at the level of accumulation of target mRNA or translation of target protein.

“Inhibition of gene expression” refers to the absence or observable decrease in the level of protein and/or mRNA product from a target gene. “Specificity” refers to the ability to inhibit the target gene without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin.

Depending on the assay, quantitation of the amount of gene expression allows one to determine a degree of inhibition which is greater than 10%, 33%, 50%, 90%, 95% or 99% as compared to a cell not treated according to the present invention. As an example, the efficiency of inhibition may be determined by assessing the amount of gene product in the cell: mRNA may be detected with a hybridization probe having a nucleotide sequence outside the region used for the inhibitory nucleic acid, or translated polypeptide may be detected with an antibody raised against the polypeptide sequence of that region.

An expression enhancing oligonucleotide as used herein is a synthetic oligonucleotide that encodes a protein. The synthetic oligonucleotide may be delivered to a cell such that it is used by a cells machinery to produce a protein based on the sequence of the synthetic oligonucleotide. The synthetic oligonucleotide may be, for instance, synthetic DNA or synthetic RNA. “Synthetic RNA” refers to a RNA produced through an in vitro transcription reaction or through artificial (non-natural) chemical synthesis. In some embodiments, a synthetic RNA is an RNA transcript. In some embodiments, a synthetic RNA encodes a protein. In some embodiments, the synthetic RNA is a functional RNA. In some embodiments, a synthetic RNA comprises one or more modified nucleotides. In some embodiments, a synthetic RNA is up to 0.5 kilobases (kb), 1 kb, 1.5 kb, 2 kb, 2.5 kb, 3 kb, 4 kb, 5 kb, 6 kb, 7 kb, 8 kb, 9 kb, 10 kb, 15 kb, 20 kb, 25 kb, 30 kb or more in length. In some embodiments, a synthetic RNA is in a range of 0.1 kb to 1 kb, 0.5 kb to 2 kb, 0.5 kb to 10 kb, 1 kb to 5 kb, 2 kb to 5 kb, 1 kb to 10 kb, 3 kb to 10 kb, 5 kb to 15 kb, or 1 kb to 30 kb in length.

A diagnostic oligonucleotide is an oligonucleotide that interacts with a cellular marker to identify the presence of the marker in a cell or subject. Diagnostic oligonucleotides are well known in the art and typically include a label or are otherwise detectable.

The terms “oligonucleotide” and “nucleic acid” are used interchangeably to mean multiple nucleotides (i.e., molecules comprising a sugar (e.g., ribose or deoxyribose) linked to a phosphate group and to an exchangeable organic base, which is either a substituted pyrimidine (e.g., cytosine (C), thymidine (T) or uracil (U)) or a substituted purine (e.g., adenine (A) or guanine (G)). Thus, the term embraces both DNA and RNA oligonucleotides. The terms shall also include polynucleosides (i.e., a polynucleotide minus the phosphate) and any other organic base containing polymer. Oligonucleotides can be obtained from existing nucleic acid sources (e.g., genomic or cDNA), but are preferably synthetic (e.g., produced by nucleic acid synthesis).

An oligonucleotide of the nanostructure can be single stranded or double stranded. A double stranded oligonucleotide is also referred to herein as a duplex. Double-stranded oligonucleotides of the invention can comprise two separate complementary nucleic acid strands.

The nucleic acids useful in the nanostructures of the invention are synthetic or isolated nucleic acids.

As used herein, “duplex” includes a double-stranded nucleic acid molecule(s) in which complementary sequences are hydrogen bonded to each other. The complementary sequences can include a sense strand and an antisense strand. The antisense nucleotide sequence can be identical or sufficiently identical to the target gene to mediate effective target gene inhibition (e.g., at least about 98% identical, 96% identical, 94%, 90% identical, 85% identical, or 80% identical) to the target gene sequence.

A double-stranded oligonucleotide can be double-stranded over its entire length, meaning it has no overhanging single-stranded sequences and is thus blunt-ended. In other embodiments, the two strands of the double-stranded polynucleotide can have different lengths producing one or more single-stranded overhangs. A double-stranded polynucleotide of the invention can contain mismatches and/or loops or bulges. In some embodiments, it is double-stranded over at least about 70%, 80%, 90%, 95%, 96%, 97%, 98% or 99% of the length of the oligonucleotide. In some embodiments, the double-stranded oligonucleotide of the invention contains at least or up to 1, 2, 3, 4, 5, 6, 7, 8, 9, 10, 11, 12, 13, 14, or 15 mismatches.

Oligonucleotides associated with the invention can be modified such as at the sugar moiety, the phosphodiester linkage, and/or the base. As used herein, “sugar moieties” includes natural, unmodified sugars, including pentose, ribose and deoxyribose, modified sugars and sugar analogs. Modifications of sugar moieties can include replacement of a hydroxyl group with a halogen, a heteroatom, or an aliphatic group, and can include functionalization of the hydroxyl group as, for example, an ether, amine or thiol.

Modification of sugar moieties can include 2′-O-methyl nucleotides, which are referred to as “methylated.” In some instances, polynucleotides associated with the invention may only contain modified or unmodified sugar moieties, while in other instances, polynucleotides contain some sugar moieties that are modified and some that are not.

In some instances, modified nucleomonomers include sugar- or backbone-modified ribonucleotides. Modified ribonucleotides can contain a non-naturally occurring base such as uridines or cytidines modified at the 5′-position, e.g., 5′-(2-amino) propyl uridine and 5′-bromo uridine; adenosines and guanosines modified at the 8-position, e.g., 8-bromo guanosine; deaza nucleotides, e.g., 7-deaza-adenosine; and N-alkylated alkylated nucleotides, e.g., N6-methyl adenosine. Also, sugar-modified ribonucleotides can have the 2′-OH group replaced by an H, alkoxy (or OR), R or alkyl, halogen, SH,

SR, amino (such as NH2, NHR, NR2,), or CN group, wherein R is lower alkyl, alkenyl, or alkynyl. In some embodiments, modified ribonucleotides can have the phosphodiester group connecting to adjacent ribonucleotides replaced by a modified group, such as a phosphorothioate group.

In some aspects, 2′-O-methyl modifications can be beneficial for reducing undesirable cellular stress responses, such as the interferon response to double-stranded nucleic acids. Modified sugars can include D-ribose, 2′-O-alkyl (including 2′-O-methyl and 2′-O-ethyl), i.e., 2′-alkoxy, 2′-amino, 2′-S-alkyl, 2′-halo (including 2′-fluoro), 2′-methoxyethoxy, 2′-allyloxy (—OCH2CH═CH2), 2′-propargyl, 2′-propyl, ethynyl, ethenyl, propenyl, and cyano and the like. The sugar moiety can also be a hexose.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. In some embodiments, a straight chain or branched chain alkyl has 6 or fewer carbon atoms in its backbone (e.g., C1-C6 for straight chain, C3-C6 for branched chain), and more preferably 4 or fewer. Likewise, preferred cycloalkyls have from 3-8 carbon atoms in their ring structure, and more preferably have 5 or 6 carbons in the ring structure. The term C1-C6 includes alkyl groups containing 1 to 6 carbon atoms.

Unless otherwise specified, the term alkyl includes both “unsubstituted alkyls” and “substituted alkyls,” the latter of which refers to alkyl moieties having independently selected substituents replacing a hydrogen on one or more carbons of the hydrocarbon backbone. The term “alkenyl” includes unsaturated aliphatic groups analogous in length and possible substitution to the alkyls described above, but that contain at least one double bond. The term alkenyl includes both “unsubstituted alkenyls” and “substituted alkenyls,” the latter of which refers to alkenyl moieties having independently selected substituents replacing a hydrogen on one or more carbons of the hydrocarbon backbone.

The term “hydrophobic modifications” refers to modification of bases such that overall hydrophobicity is increased and the base is still capable of forming close to regular Watson-Crick interactions. Non-limiting examples of base modifications include 5-position uridine and cytidine modifications like phenyl, 4-pyridyl, 2-pyridyl, indolyl, and isobutyl, phenyl (C₆H₅OH); tryptophanyl (C₈H₆N)CH₂CH(NH₂)CO), Isobutyl, butyl, aminobenzyl; phenyl; and naphthyl.

The term “base” includes the known purine and pyrimidine heterocyclic bases, deazapurines, and analogs (including heterocyclic substituted analogs, e.g., aminoethyoxy phenoxazine), derivatives (e.g., 1-alkyl-, 1-alkenyl-, heteroaromatic- and 1-alkynyl derivatives) and tautomers thereof. Examples of purines include adenine, guanine, inosine, diaminopurine, and xanthine and analogs (e.g., 8-oxo-N6-methyladenine or 7-diazaxanthine) and derivatives thereof. Pyrimidines include, for example, thymine, uracil, and cytosine, and their analogs (e.g., 5-methylcytosine, 5-methyluracil, 5-(1-propynyl)uracil, 5-(1-propynyl)cytosine and 4,4-ethanocytosine). Other examples of suitable bases include non-purinyl and non-pyrimidinyl bases such as 2-aminopyridine and triazines.

In some aspects, polynucleotides of the invention comprise 3′ and 5′ termini (except for circular oligonucleotides). The 3′ and 5′ termini of a polynucleotide can be substantially protected from nucleases, for example, by modifying the 3′ or 5′ linkages (e.g., U.S. Pat. No. 5,849,902 and WO 98/13526). Oligonucleotides can be made resistant by the inclusion of a “blocking group.” The term “blocking group” as used herein refers to substituents (e.g., other than OH groups) that can be attached to oligonucleotides or nucleomonomers, either as protecting groups or coupling groups for synthesis (e.g., FITC, propyl (CH₂-CH₂-CH₃), glycol (—O—CH₂—CH₂—O—) phosphate, hydrogen phosphonate, or phosphoramidite). “Blocking groups” also include “end blocking groups” or “exonuclease blocking groups” which protect the 5′ and 3′ termini of the oligonucleotide, including modified nucleotides and non-nucleotide exonuclease resistant structures.

Exemplary end-blocking groups include cap structures (e.g., a 7-methylguanosine cap), inverted nucleomonomers, e.g., with 3′-3′ or 5′-5′ end inversions (see, e.g., Ortiagao et al. 1992. Antisense Res. Dev. 2:129), methylphosphonate, phosphoramidite, non-nucleotide groups (e.g., non-nucleotide linkers, amino linkers, conjugates) and the like. The 3′ terminal nucleomonomer can comprise a modified sugar moiety. The 3′ terminal nucleomonomer comprises a 3′-O that can optionally be substituted by a blocking group that prevents 3′-exonuclease degradation of the oligonucleotide. For example, the 3′-hydroxyl can be esterified to a nucleotide through a 3′→3′ internucleotide linkage. For example, the alkyloxy radical can be methoxy, ethoxy, or isopropoxy, and preferably, ethoxy. Optionally, the 3′→31inked nucleotide at the 3′ terminus can be linked by a substitute linkage. To reduce nuclease degradation, the 5′ most 3′→5′ linkage can be a modified linkage, e.g., a phosphorothioate or a P-alkyloxyphosphotriester linkage. Preferably, the two 5′ most 3′→5′ linkages are modified linkages. Optionally, the 5′ terminal hydroxy moiety can be esterified with a phosphorus containing moiety, e.g., phosphate, phosphorothioate, or P-ethoxyphosphate.

The term “nucleoside” includes bases which are covalently attached to a sugar moiety, preferably ribose or deoxyribose. Examples of preferred nucleosides include ribonucleosides and deoxyribonucleosides. Nucleosides also include bases linked to amino acids or amino acid analogs which may comprise free carboxyl groups, free amino groups, or protecting groups. Suitable protecting groups are well known in the art (see P.

G. M. Wuts and T. W. Greene, “Protective Groups in Organic Synthesis”, 2nd Ed., Wiley-Interscience, New York, 1999).

The nanostructures of the invention contemplate the use of linkers. The linkers may be linkers between the hydrophobic molecule and other therapeutic or diagnostic molecules. The linkers may also be nucleic acid linkers between nucleic acids, including standard phosphodiester internucleotide linkages as well as modified internucleotide linkages. The linkers may also be non-standard linkages that link hydrophobic molecules with nucleic acids or with other compounds such as proteins. As used herein, the term nucleotide linkage includes a naturally occurring, unmodified phosphodiester moiety (—O—(PO2—)—O—) that covalently couples adjacent nucleomonomers as well as any analog or derivative of the native phosphodiester group that covalently couples adjacent nucleomonomers. Analogs or derivatives include phosphodiester analogs, e.g., phosphorothioate, phosphorodithioate, and P-ethyoxyphosphodiester, P-ethoxyphosphodiester, P-alkyloxyphosphotriester, methylphosphonate, phosphoramidates, thio-phosphoramidates, and nonphosphorus containing linkages, e.g., acetals and amides. Such substitute linkages are known in the art (e.g., Bjergarde et al. 1991. Nucleic Acids Res. 19:5843; Caruthers et al. 1991. Nucleosides Nucleotides. 10:47).

A non-nucleotidic linker or spacer sequence may be a peptide, a lipid, a polymer or an oligoethylene. Examples of linkers or spacers of the invention include HEG and PEG.

In an embodiment containing a liposomal core, the oligonucleotide shell may be anchored to the surface of the liposomal core through conjugation to one or a multiplicity of linker molecules including but not limited to: tocopherols, sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and their derivatives.

In some embodiments at least 25, 50, 75, 100, 200, 300, 400, 500, 600, 700, 800, 900, 1,000, 2,000, 3,000, 4,000, 5,000, 6,000, 7,000, 8,000, 9,000 or 10,000 oligonucleotides or any range combination thereof are on the exterior of the core. In some embodiments, 1-10,000, 1-9,000, 1-8,000, 1-7,000, 1-6,000, 1-5,000, 1-4,000, 1-3,000, 1-2,000, 1-1,000, 5-10,000, 5-9,000, 5-8,000, 5-7,000, 5-6,000, 5-5,000, 5-4,000, 5-3,000, 5-2,000, 5-1,000, 100-10,000, 100-9,000, 100-8,000, 100-7,000, 100-6,000, 100-5,000, 100-4,000, 100-3,000, 100-2,000,100-1,000, 500-10,000, 500-9,000, 500-8,000, 500-7,000, 500-6,000, 500-5,000, 500-4,000, 500-3,000, 500-2,000, 500-1,000, 10-10,000, 10-500, 50-10,000, 50-300, or 50-250 oligonucleotides are present on the surface of the nanostructure.

In some embodiments, the oligonucleotides of the oligonucleotide shell are structurally identical oligonucleotides. In other embodiments, the oligonucleotides of the oligonucleotide shell have at least two structurally different oligonucleotides. In certain embodiments, the oligonucleotides of the oligonucleotide shell have 2-50, 2-40, 2-30, 2-20 or 2-10 different nucleotide sequences.

In some embodiments, at least 60%, 70%, 80%, 90%, 95%, 96%, 97% 98% or 99% of the oligonucleotides are positioned on the surface of the nanostructure.

In some embodiments, the oligonucleotides form an oligonucleotide shell. An oligonucleotide shell is formed when at least 10% of the available surface area of the exterior surface of a liposomal core includes an oligonucleotide. In some embodiments at least 20%, 30%, 40%, 50%, 60%, 70%, 80%, 90%, 95%, 96%, 97%, 98% or 99% of the available surface area of the exterior surface of the liposomal includes an oligonucleotide. The oligonucleotides of the oligonucleotide shell may be oriented in a variety of directions. In some embodiments the oligonucleotides are oriented radially outwards.

In some embodiments, at least 10% of the oligonucleotides in the oligonucleotide shell are attached to the nanoparticle through a lipid anchor group. The lipid anchor consists of a hydrophobic group that enables insertion and anchoring of the oligonucleotides or nucleic acids to the lipid membrane. In some embodiments, at least 20%, at least 30%, at least 40%, at least 50%, at least 60%, at least 70%, at least 80%, at least 90%, at least 95%, at least 99%, or 100% of the oligonucleotides in the oligonucleotide shell are attached to the lipid nanoparticle through a lipid anchor group. In some embodiments, the lipid anchor group is cholesterol. In other embodiments, the lipid anchor group is sterol, palmitoyl, dipalmitoyl, stearyl, distearyl, C16 alkyl chain, bile acids, cholic acid, taurocholic acid, deoxycholate, oleyl litocholic acid, oleoyl cholenic acid, glycolipids, phospholipids, sphingolipids, isoprenoids, such as steroids, vitamins, such as vitamin E, saturated fatty acids, unsaturated fatty acids, fatty acid esters or other lipids known in the art.

In some embodiments, the oligonucleotides have a spacer between the oligonucleotide and the lipid anchor group. A non-limiting example of a spacer is hexa(ethylene)glycol.

In some embodiments, the liposome to hydrophobic molecule ratio (mass/mass ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1, or about 6:1, 7:1, 8:1, 9:1, 10:1, 11:1, 12:1, 33:1, 92:8, 95:5, 97:3 or 99:1.

As used herein, the nanostructure is a construct having an average diameter on the order of nanometers (i.e., between about 1 nm and about 1 micrometer. For example, in some instances, the diameter of the nanoparticle is from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 ran in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, about 1 nm to about 20 nm in mean diameter, about 1 nm to about 10 nm in mean diameter, about 5 nm to about 150 nm in mean diameter, about 5 to about 50 nm in mean diameter, about 10 to about 30 nm in mean diameter, about 10 to 150 nm in mean diameter, about 10 to about 100 nm in mean diameter, about 10 to about 50 nm in mean diameter, about 30 to about 100 nm in mean diameter, or about 40 to about 80 nm in mean diameter.

Aspects of the invention relate to delivery of nanostructures to a subject for therapeutic and/or diagnostic use. The nanostructure may be administered alone or in any appropriate pharmaceutical carrier, such as a liquid, for example saline, or a powder, for administration in vivo. They can also be co-delivered with larger carrier particles or within administration devices. The nanostructure may be formulated. The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients. In some embodiments, nanostructures associated with the invention are mixed with a substance such as a lotion (for example, aquaphor) and are administered to the skin of a subject, whereby the nanostructures are delivered through the skin of the subject. It should be appreciated that any method of delivery of nanoparticles known in the art may be compatible with aspects of the invention.

The nanostructures of the invention result in more effective therapies for prophylactic or therapeutic uses in treating a wide variety of diseases/infections including, for example, AIDS, malaria, chlamydia, campylobacter, cytomegalovirus, dengue, Epstein-Barr mononucleosis, foot and mouth disease, rabies, Helicobacter pylori gastric ulcers, hepatitis A, B, C, herpes simplex, influenza, leishmaniasis, cholera, diphtheria, Haemophilus influenza, meningococcal meningitis, plague, pneumococcal pneumonia, tetanus, typhoid fever, respiratory synctial virus, rhinovirus, schistosomiasis, shigella, streptococcus group A and B, tuberculosis, vibrio cholera, salmonella, aspergillus, blastomyces, histoplasma, candida, cryptococcus, pneumocystis, and urinary tract infections; various food allergies such as peanut, fruit, garlic, oats, meat, milk, fish, shellfish, soy, tree nut, wheat, gluten, egg, sulphites; various drug allergies such as to tetracycline, Dilantin, carbamazepine, penicillins, cephalosporins, sulfonamides, NSAIDs, intravenous contrast dye, local anesthetics; autoimmune diseases such as multiple sclerosis, lupus, inflammatory bowel disease, Crohn's disease, ulcerative colitis, asthma, and COPD; and cancers such as melanoma, breast cancer, prostate cancer, bladder cancer, NSCLC, glioblastoma multiforme, among others.

For use in therapy, an effective amount of the nanostructure can be administered to a subject by any mode that delivers the nanostructure to the desired cell. Administering pharmaceutical compositions may be accomplished by any means known to the skilled artisan. Routes of administration include but are not limited to oral, parenteral, intramuscular, intravenous, subcutaneous, mucosal, intranasal, sublingual, intratracheal, inhalation, ocular, vaginal, dermal, rectal, and by direct injection.

Thus the nanostructure is useful in some aspects of the invention as a stand-alone therapeutic, in combination with other therapeutics, or as a vaccine for the treatment of a subject at risk of developing or a subject having allergy or asthma, an infection with an infectious organism or a cancer. The nanostructure may also include an antigen or allergen or be delivered together with an antigen or allergen for protection against infection, allergy or cancer, and in this case repeated doses may allow longer term protection. A subject at risk, as used herein, is a subject who has any risk of exposure to an infection causing pathogen or a cancer or an allergen or a risk of developing cancer. For instance, a subject at risk may be a subject who is planning to travel to an area where a particular type of infectious agent is found or it may be a subject who through lifestyle or medical procedures is exposed to bodily fluids which may contain infectious organisms or directly to the organism or even any subject living in an area where an infectious organism or an allergen has been identified. Subjects at risk of developing infection also include general populations to which a medical agency recommends vaccination with a particular infectious organism antigen. If the antigen is an allergen and the subject develops allergic responses to that particular antigen and the subject may be exposed to the antigen, i.e., during pollen season, then that subject is at risk of exposure to the antigen.

A subject having an infection is a subject that has been exposed to an infectious pathogen and has acute or chronic detectable levels of the pathogen in the body. The nanostructure having immunostimulatory properties i.e., hydrophobic molecules or oligonucleotides can be used with or without an antigen to mount an antigen specific systemic or mucosal immune response that is capable of reducing the level of or eradicating the infectious pathogen. An infectious disease, as used herein, is a disease arising from the presence of a foreign microorganism in the body. It is particularly important to develop effective vaccine strategies and treatments to protect the body's mucosal surfaces, which are the primary site of pathogenic entry.

A subject having an allergy is a subject that has or is at risk of developing an allergic reaction in response to an allergen. An allergy refers to acquired hypersensitivity to a substance (allergen). Allergic conditions include but are not limited to eczema, allergic rhinitis or coryza, hay fever, conjunctivitis, bronchial asthma, urticaria (hives) and food allergies, and other atopic conditions.

A subject having a cancer is a subject that has detectable cancerous cells. The cancer may be a malignant or non-malignant cancer. Cancers or tumors include but are not limited to biliary tract cancer; brain cancer; breast cancer; cervical cancer; choriocarcinoma; colon cancer; endometrial cancer; esophageal cancer; gastric cancer; intraepithelial neoplasms; lymphomas; liver cancer; lung cancer (e.g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas. In one embodiment the cancer is hairy cell leukemia, chronic myelogenous leukemia, cutaneous T-cell leukemia, multiple myeloma, follicular lymphoma, malignant melanoma, squamous cell carcinoma, renal cell carcinoma, prostate carcinoma, bladder cell carcinoma, or colon carcinoma.

As used herein, the term treat, treated, or treating when used with respect to an disorder such as an infectious disease, cancer, allergy, autoimmune disease or asthma refers to a prophylactic treatment which increases the resistance of a subject to development of the disease (e.g., to infection with a pathogen) or, in other words, decreases the likelihood that the subject will develop the disease (e.g., become infected with the pathogen) as well as a treatment after the subject has developed the disease in order to fight the disease (e.g., reduce or eliminate the infection) or prevent the disease from becoming worse.

An antigen as used herein is a molecule capable of provoking an immune response. Antigens include but are not limited to cells, cell extracts, proteins, polypeptides, peptides, polysaccharides, polysaccharide conjugates, peptide and non-peptide mimics of polysaccharides and other molecules, small molecules, lipids, glycolipids, carbohydrates, viruses and viral extracts and muticellular organisms such as parasites and allergens. The term antigen broadly includes any type of molecule which is recognized by a host immune system as being foreign. Antigens include but are not limited to cancer antigens, microbial antigens, and allergens.

A cancer antigen as used herein is a compound, such as a peptide or protein, associated with a tumor or cancer cell surface and which is capable of provoking an immune response when expressed on the surface of an antigen presenting cell in the context of an MHC molecule. Cancer antigens can be prepared from cancer cells either by preparing crude extracts of cancer cells, for example, as described in Cohen, et al., 1994, Cancer Research, 54:1055, by partially purifying the antigens, by recombinant technology, or by de novo synthesis of known antigens. Cancer antigens include but are not limited to antigens that are recombinantly expressed, an immunogenic portion of, or a whole tumor or cancer. Such antigens can be isolated or prepared recombinantly or by any other means known in the art.

A microbial antigen as used herein is an antigen of a microorganism and includes but is not limited to virus, bacteria, parasites, and fungi. Such antigens include the intact microorganism as well as natural isolates and fragments or derivatives thereof and also synthetic compounds which are identical to or similar to natural microorganism antigens and induce an immune response specific for that microorganism. A compound is similar to a natural microorganism antigen if it induces an immune response (humoral and/or cellular) to a natural microorganism antigen. Such antigens are used routinely in the art and are well known to those of ordinary skill in the art.

The nanostructures are also useful for treating and preventing autoimmune disease. Autoimmune disease is a class of diseases in which an subject's own antibodies react with host tissue or in which immune effector T cells are autoreactive to endogenous self peptides and cause destruction of tissue. Thus an immune response is mounted against a subject's own antigens, referred to as self antigens. Autoimmune diseases include but are not limited to rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjogren's syndrome, insulin resistance, and autoimmune diabetes mellitus.

The nanostructure can be combined with other therapeutic agents. The nanostructure and/or other therapeutic agent may be administered simultaneously or sequentially. When the other therapeutic agents are administered simultaneously they can be administered in the same or separate formulations, but are administered at the same time. The other therapeutic agents are administered sequentially with one another and with the nanostructure, when the administration of the other therapeutic agents and the nanostructure is temporally separated. The separation in time between the administration of these compounds may be a matter of minutes or it may be longer.

The term “effective amount” of a nanostructure refers to the amount necessary or sufficient to realize a desired biologic effect. For example, an effective amount of a nanostructure is that amount necessary to elicit an improved cytokine response across multiple cytokines. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular nanostructure being administered the size of the subject, or the severity of the disease or condition. One of ordinary skill in the art can empirically determine the effective amount of a particular nanostructure without necessitating undue experimentation.

Subject doses of the compounds described herein typically range from about 0.1 μg to 10,000 mg, more typically from about 1 μg /day to 8000 mg, and most typically from about 10 μg to 100 μg. Stated in terms of subject body weight, typical dosages range from about 0.1 μg to 20 mg/kg/day, more typically from about 1 to 10 mg/kg/day, and most typically from about 1 to 5 mg/kg/day.

In other embodiments of the invention, the nanostructure is administered on a routine schedule. A “routine schedule” as used herein, refers to a predetermined designated period of time. The routine schedule may encompass periods of time which are identical or which differ in length, as long as the schedule is predetermined. For instance, the routine schedule may involve administration of the nanostructure on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc. Alternatively, the predetermined routine schedule may involve administration of the nanostructure on a daily basis for the first week, followed by a monthly basis for several months, and then every three months after that. Any particular combination would be covered by the routine schedule as long as it is determined ahead of time that the appropriate schedule involves administration on a certain day.

As used herein, a “subject” or a “patient” refers to any mammal (e.g., a human), for example, a mammal that may be susceptible to a disease, disorder or bodily condition. Examples of subjects or patients include a human, a non-human primate, a cow, a horse, a pig, a sheep, a goat, a dog, a cat or a rodent such as a mouse, a rat, a hamster, or a guinea pig. Generally, the invention is directed toward use with humans. A subject may be a subject diagnosed with a certain disease or bodily condition or otherwise known to have a disease or bodily condition. In some embodiments, a subject may be diagnosed as, or known to be, at risk of developing a disease or bodily condition. In certain embodiments, a subject may be selected for treatment on the basis of a known disease or bodily condition in the subject. In some embodiments, a subject may be selected for treatment on the basis of a suspected disease or bodily condition in the subject. In some embodiments, the composition may be administered to prevent the development of a disease or bodily condition. However, in some embodiments, the presence of an existing disease or bodily condition may be suspected, but not yet identified, and a composition of the invention may be administered to diagnose or prevent further development of the disease or bodily condition.

A “biological sample,” as used herein, is any cell, body tissue, or body fluid sample obtained from a subject. Non-limiting examples of body fluids include, for example, lymph, saliva, blood, urine, and the like. Samples of tissue and/or cells for use in the various methods described herein can be obtained through standard methods including, but not limited to, tissue biopsy, including punch biopsy and cell scraping, needle biopsy; or collection of blood or other bodily fluids by aspiration or other suitable methods.

This application refers to various issued patents, published patent applications, journal articles, and other publications, all of which are incorporated herein by reference.

If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

### EXAMPLES

### Example 1.

### Functionalization of Glycopyranoside Lipid A (GLA) to the Surface of Liposomes and Liposomal Spherical Nucleic Acid (SNA) Constructs

We describe a method of selectively functionalizing lipophilic and/or hydrophobic molecules to the surface of liposomes and liposomal spherical nucleic acids (SNA). Typically, liposomes are formed by extrusion. After undergoing extrusion, or other method of liposome synthesis, the hydrophobic molecules will be distributed throughout the liposomal unilamellar membrane, likely buried within the bilayer, resulting in low activity. When hydrophobic molecules such as glycopyranoside lipid A (GLA) need to be conjugated to liposomal SNAs their efficacy in specific toll-like receptor (TLR) stimulation is affected by their presentation on the SNA surface. In order to surface functionalize a highly hydrophobic molecule such as GLA, which is a synthetic form of natural monophosphoryl Lipid A found in E coli that specifically agonizes TLR4, to the outer surface of the liposomal SNA such that it retains its efficient activity as a TLR stimulating molecule, we have developed a method to synthesize liposomal SNAs with externally functionalized GLA (L-GLA) in an aqueous solution. The specific activity of GLA as a TLR4 stimulating molecule is retained after its functionalization to the surface of the liposomal SNA. Also, the ability of co-loading other TLR agonists (TLR9/8/7) will open the door for improved cytokine response across multiple cytokines.

### Materials and Methods

### Liposome Synthesis

Liposomes were synthesized by extrusion of 1,2-dioleoyl-sn-glycero-3-phosphocholine (DOPC) hydrated in phosphate buffered saline solution (PBS) (137 mM NaCl, 10 mM phosphate, 2.7 mM KC1, pH 7.4, hyclone) using 47 mm diameter polycarbonate membranes with 50 nm pores (Sterlitech). Liposome diameters were measured using dynamic light scattering using a Malvern Zetasizer Nano (Malvern Instruments). Lipid concentration was determined using a phospholipid assay kit (Sigma).

### Surface Functionalized GLA Liposome (L-GLA) Synthesis

GLA (Avanti Polar Lipids) was resuspended in dimethylsulfoxide (DMSO) at 1 mg/mL. GLA in DMSO was mixed with an aqueous solution of liposomes (L-GLA) or PBS (GLA alone) such that the final DMSO concentration was 2%. DMSO is miscible in aqueous solutions, and allows GLA to condense on the surface of SNAs as the organic solution is diluted. Modulation of GLA functionalized to the surface of liposomes can be achieved by adjusting the ratio of GLA to liposome. The DMSO was removed from the liposomal solution by diafiltration using tangential-flow filtration (TFF) with a hollow fiber filter (750 kDa cut-off). These liposomes which have been surface functionalized with GLA (with a loading of 50 molecules/liposome) may then be further surface modified with other molecules. L-GLA or GLA alone was then sterile filtered through a 0.22 μm PES sterile syringe filter (VWR) to remove any non-functionalized GLA or GLA aggregates.

### Co-extruded GLA Liposome (L-GLA) Synthesis

DOPC was solubilized in dichloromethane (100 mg/mL). GLA was solubilized in dichloromethane (1 mg/mL). Mixtures of DOPC and GLA made at 99:1, 97:3, 95:5, and 92:8 (% weight) and the mixtures were dried as a thin film in sterile, pyrogen free glass vials. DOPC/GLA films were hydrated in PBS and liposomes were synthesized by extrusion using 47 mm diameter polycarbonate membranes with 50 nm pores (Sterlitech). Liposome diameters were measured using dynamic light scattering using a Malvern Zetasizer Nano (Malvern Instruments). Lipid concentration was determined using a phospholipid assay kit (Sigma).

### Multi-ligand Spherical Nucleic Acid Structure (Multi-ligand SNA)

Multi-ligand SNAs are composed of L-GLA (TLR4 agonist), in addition to oligo 6589 (TLR9 agonist) or oligo 7046 (TLR7/8 agonist), which are functionalized on to L-GLA. GLA concentration on these multi-ligand SNA structures can be altered to produce a desired cytokine profile.

TABLE 1
TLR agonist.

| Oligo | SEQ ID | |
|---|---|---|
| ID | NO | 5′→3′ |
| 6589 | 1 | T*C*G*T*C*G*T*T*T*T*G*T*C*G*T*T*T*T*G* |
| | | T*C*G*T*T/iSp18//iSp18/Chol/ |
| 7046 | 2 | /Chol//iSp18//iSp18/ |
| | | rU*rU*rG*rU*rU*rG*rU*rU*rG*rU*rU*rG* |
| | | rU*rU*rG*rU*rU*rG*rU*rU |

Oligo 6589 is a DNA sequence composed of phosphothioate backbone and 4 CpG motifs.
In addition, a 3′ cholesterol anchor was attached using 2 spacer 18 linkages.
Oligo 7046 is a RNA sequence composed of a phosphothioate backbone and 6-repeat UUG sequence.
A 5′ cholesterol anchor was attached using 2 spacer 18 linkages.
“*” denotes a phosphorothioate bond,
“/iSp18/” denotes a hexa(ethylene glycol)spacer,
“/Chol/” denotes a cholesterol phosphoroamidite

In the following studies, multi-ligand SNAs were created using L-GLA (with a loading of 50 molecules/liposome). Sample preparation for multi-ligand experiments consisted of 4 stocks of L-GLA and oligo mix for each multi-ligand compound. All concentrations were made at 10× concentration.

For the oligo 6589 multi-ligand SNA, 4 stocks of L-GLA were created at 6.25 μM, 1.56 μM, 0.39 μM and 0.10 μM concentration. A consistent 0.15 μM concentration of oligo 6589 was added to each L-GLA stock. The oligo 7046 multi-ligand SNA consisted of 4 stocks of L-GLA at 12.5 μM, 3.13 μM, 0.78 μM and 0.20 μM concentration. A consistent 1.56 μM concentration of oligo 7046 was added to each L-GLA stock.

### Cell Culture

For experiment 1, HEK Blue huTLR4 cell lines were purchased from Invivogen.

HEK huTLR4 culture (growth) medium was composed of DMEM, 4.5 g/1 glucose, 10% (v/v) fetal bovine serum, 50 U/mL penicillin, 50 μg/mL streptomycin, 100 μg/mL Normocin™, 100 μg/mL Zeocin™, 10 μg/mL Blasticidin, 2 mM L-glutamine. Cell cultures were stored in T75 flasks (Nonpyrogenic polystyrene) from Corning at 37° C. and 5% CO₂.

For PBMC culture (experiment 2 and 3), quarter sized Leukopaks, which were collected by apheresis, were purchased from Stemcell Technologies. Leukopaks were further processed using ammonium chloride to lyse and remove red blood cells. PBMC were cultured in RPMI (RPMI with Phenol Red (Corning), 4.5 g/1 glucose, 10% (v/v) fetal bovine serum, 50 U/ml penicillin, and 50 mg/ml streptomycin) and were allowed to rest overnight at 37 ° C. and 5% CO₂ before use the following day. Medium was replaced prior to treatment.

Cell Immunostimulation

For experiment 1, Liposomes synthesized with 1%, 3%, 5%, and 8% GLA by co-extrusion with GLA, or surface functionalized with GLA, were added to HEK Blue huTLR4 cells in a 96 well plate at 20.0 nM, 4.0 nM, and 0.1 nM liposomes concentrations. Cells were incubated for 16 hours at 37 ° C. Specific TLR4 immunostimulation was assayed using the QuantiBlue method as described below.

For experiment 2 and 3, RPMI media was exchanged with fresh RPMI media prior to immunostimulation. L-GLA compounds were prepared at 10× concentrations, and 20 μL L-GLA, which was co-loaded with either oligo 6589 or oligo 7046, was added to 180 μL of cells (2 million cells/well). Treated cells were placed in the incubator at 37° C. and 5% CO₂ for approximately 24 hours before removing supernatant for cytokine analysis using a Q-Plex Chemiluminescent array (Quansys).

For experiment 4, GLA alone and L-GLA (pre- and post-sterile filtration) were added to HEK huTLR4 cells in a 96 well plate at a maximum concentration of 15 μM with 1:3 serial dilution for 7 dilutions. Cells were incubated for 24 hours at 37° C. Specific TLR4 immunostimulation was assayed using the QuantiBlue method as described below.

### QuantiBlue

QuantiBlue detection media was purchased from Invivogen. 160 μL of QuantiBlue was added to each well of a sterile 96-well plate, and 40 μL of cell supernatant was added to their corresponding well to obtain a total volume of 200 μL. Once all the test compounds were plated, the plates were placed in an incubator at 37° C. and 5% CO₂ for 30 minutes. Color progression was checked every 15 minutes after the 30-minute incubation period. After development of color using the standard curve as a reference, the plate was read using a fluorescence plate reader (Synergy 4) at an absorbance of 650 nm.

### Cytokine Analysis

A standard curve was prepared using sample diluent, which was provided in the kit. The supernatant collected from the transfected cells were diluted 1:2 using sample diluent. 50 μL of standard and samples were added to the Q-Plex 96-well plate. The plate was sealed and placed on the shaker (500 rpm and 20° C.) for 1 hour. The plate was then washed 3 times with wash buffer. 50 μL of Detection mix was added to each well.

Again, the plate was sealed and placed on shaker (500 rpm and 20° C.) for 1 hour. The plate was then washed 3 more times. 50 μL of Streptavidin-HRP 1× was added to each well, and the plate was sealed and returned to the shaker (500 rpm and 20° C.) for 15 minutes. During this time mixed substrate was prepared, taking care to protect it from UV light. The plate was then washed 6 times. 50 μL of substrate mix were added to each well, and the plate was read using a Bio-Rad ChemiDoc XRS+ imager within 15 minutes.

### Results

### Experiment 1

Liposomes with MPLA/GLA added to the surface after extrusion using the methods described herein exhibit greater stimulation of TLR 4 cell lines than liposomes that have MPLA/GLA co-extruded at the same MPLA/GLA concentration. These results demonstrate that MPLA/GLA has greater activity when surface functionalized to liposomes versus when it is co-extruded with the liposome.

### Experiment 2

L-GLA treatment induced dose-independent release of Thl promoters (TNFα, IL-6 and IP-10 cytokines) and Th2 promoters (IL-10), as well as dose-dependent release of IL-1β Th1 promoter) and a low dose-dependent release of IL-4 (Th2 promoter). The results varied somewhat between the two donors.

### Experiment 3

Multi-ligand SNA treatment with increasing GLA concentration induced dose-dependent release of Th1 promoters (IL-lα, IL-6 and IFN-γ) and Th2 promoters (IL-4, IL-5, and IL-10) and cytokines, as well as low dose-dependent release of IL-β (Th1 promoter). A dose-independent release of TNF-α(Th1 promoter) was observed in both instances.

### Experiment 4

GLA, when surface functionalized onto DOPC liposomes, was able to activate HEK hTLR4 cells post-sterile filtration. GLA alone was prepared using the same method used to prepare L-GLA but without the presence of liposomes. Free or unbound GLA will aggregate and cannot pass through a sterile filter. This is demonstrated by the result that GLA alone failed to activate HEK hTLR4, indicating free GLA is removed by sterile filtration. GLA that surface functionalizes into the 50 nm liposomes is able to pass through a 0.22 μm sterile filter and activates HEK hTLR4 cells post-sterile filtration.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section

1. A method of functionalizing a hydrophobic molecule to the surface of a liposome, comprising
mixing an aqueous solution of a liposome with a hydrophobic molecule in an organic solvent to produce a liposomal solution and removing the organic solvent from the liposomal solution to produce a nanostructure comprised of the liposome surface functionalized with the hydrophobic molecule.

2. The method of claim 1, wherein the liposome is mixed with the hydrophobic molecule in a ratio of 99:1, 97:3, 95:5, or 92:8 by percent weight.

3. (canceled)

4. The method of claim 1, further comprising mixing the nanostructure with an oligonucleotide.

5. The method of claim 4, wherein at least 95% of the oligonucleotides are positioned on the surface of the nanostructure.

6.-13. (canceled)

14. The method of claim 4, wherein the oligonucleotide is a toll-like receptor 9 (TLR9) agonist.

15. The method of claim 14, wherein the TLR9 receptor agonist is a CpG oligonucleotide.

16.-17. (canceled)

18. A nanostructure, comprising a liposome having at least 40 hydrophobic molecules functionalized on the surface of the liposome.

19. The nanostructure of claim 18, wherein 40-60 hydrophobic molecules are functionalized on the surface of the liposome, 45-55 hydrophobic molecules are functionalized on the surface of the liposome, or 50 hydrophobic molecules are functionalized on the surface of the liposome.

20.-21. (canceled)

22. The nanostructure of claim 18, wherein at least 60% of the hydrophobic molecules in the nanostructure are positioned on the surface of the liposome, at least 70% of the hydrophobic molecules in the nanostructure are positioned on the surface of the liposome, at least 80% of the hydrophobic molecules in the nanostructure are positioned on the surface of the liposome, at least 90% of the hydrophobic molecules in the nanostructure are positioned on the surface of the liposome, or 100% of the hydrophobic molecules in the nanostructure are positioned on the surface of the liposome.

23.-26. (canceled)

27. The nanostructure of claim 18, wherein the liposome further comprises an oligonucleotide shell on the surface of the liposome.

28. The nanostructure of claim 27, wherein the oligonucleotide shell is comprised of oligonucleotides, wherein at least 95% of the oligonucleotides are attached to the lipid nanoparticle through a lipid anchor group.

29.-32. (canceled)

33. The nanostructure of claim 28, wherein the oligonucleotide is a toll-like receptor 9 (TLR9) agonist.

34. The nanostructure of claim 33, wherein the TLR9 receptor agonist is a CpG oligonucleotide.

35.-41. (canceled)

42. The nanostructure of claim 28, wherein the oligonucleotides are toll-like receptor 7/8 (TLR7/8) agonists.

43.-46. (canceled)

47. The nanostructure of claim 28, wherein the oligonucleotides include at least two structurally different oligonucleotides.

48. (canceled)

49. The nanostructure of claim 27, wherein the oligonucleotide shell has a density of 5-1,000 oligonucleotides per nanostructure a density of 100-1,000 oligonucleotides per nanostructure, or a density of 500-1,000 oligonucleotides per nanostructure.

50.-51. (canceled)

52. The nanostructure of claim 28, wherein the oligonucleotides have 5′-termini exposed to the outside surface of the nanostructure or the oligonucleotides have 3′-termini exposed to the outside surface of the nanostructure.

53. (canceled)

54. A method for treating a disease or disorder in a subject, comprising administering to the subject a nanostructure of claim 18 to elicit an immune response and treat the disease or disorder.

55. The method of claim 54, wherein the disease or disorder is cancer, asthma, infection, or allergy.

56.-58. (canceled)

59. The method of claim 54, wherein the subject is a mammal.

60. (canceled)
